(19) United States
(12) Patent Application Publication
(10) Pub. No.: US 2022/0369966 A1
(43) Pub. Date: Nov. 24, 2022

(54) BIOLOGICAL INFORMATION COLLECTION SENSOR UNIT, BIOLOGICAL INFORMATION COLLECTION DEVICE, AND BIOLOGICAL INFORMATION PROCESSING UNIT

(71) Applicants: ASTEM Co., Ltd., Kawasaki-shi, Kanagawa (JP); National University Corporation Hamamatsu University School of Medicine, Hamamatsu-shi, Shizuoka (JP)

(72) Inventors: Hikaru SUZUKI, Kawasaki-shi, Kanagawa (JP); Naoki UNNO, Hamamatsu-shi, Shizuoka (JP)

(21) Appl. No.: 17/761,930

(22) PCT Filed: Oct. 21, 2020
(86) PCT No.: PCT/JP2020/039614

§ 371 Date: Mar. 18, 2022

(30) Foreign Application Priority Data
Nov. 12, 2019 (JP) 2019-204705

(51) Int. Cl. A61B 5/1455; A61B 5/00

(52) U.S. Cl. CPC A61B 5/14552; A61B 5/6829; A61B 5/6828; A61B 5/742; A61B 2562/04

(57) ABSTRACT

A biological information collection sensor unit includes sensors and a processor. Each sensor has a light emitter emitting near-infrared or infrared light and a light receiver collecting transmitted light including at least light transmitting through a measurement target tissue. The sensors are arranged on surfaces of a measurement target region and configured to collect optical information regarding tissues of the measurement target region. The processor calculates tissue oxygen saturation information regarding oxygen saturation of the tissue based on the optical information. The sensors are arranged in different areas of the measurement target region. The processor includes an arithmetic processor to calculate the tissue oxygen saturation information for each sensor and a notifier to output a notification signal when the number of the sensors collecting the optical information used to calculate the tissue oxygen saturation information equal to or greater than a preset threshold satisfies a specified condition.

### CROSS-REFERENCE TO RELATED APPLICATION

This international application claims the benefit of Japanese Patent Application No. 2019-204705, filed on Nov. 12, 2019 with the Japan Patent Office, and the entire disclosure of Japanese Patent Application No. 2019-204705 is incorporated herein by reference.

### TECHNICAL FIELD

The present disclosure relates to a biological information collection sensor unit, a biological information collection device, and a biological information processing unit that are suitable for use in measurement of biological information, such as transcutaneous tissue oxygen saturation.

### BACKGROUND ART

For example, as disclosed in Patent Document 1, there is a known device for measurement of transcutaneous oxygen partial pressure (TcPO2) in order to recognize a state of blood perfusion in a lower limb of a patient suffering from a disease, such as a patient with peripheral arterial occlusive disease or a patient with arteriosclerosis obliterans. This is a device to heat a skin of a patient subject to measurement and measure oxygen partial pressure of the skin using an electrochemical sensor that can obtain a transcutaneously measured value of a blood gas. The transcutaneous oxygen partial pressure (TcPO2) is known to have a good relationship with oxygen partial pressure of arterial blood (PaO2) in heated subcutaneous tissue. Thus, the device is used for, for example, diagnosing a condition of local tissue blood flow.

In a field such as plastic surgery, a device is desired that is able to measure a blood flow condition of a transplanted tissue in real time, in order to determine whether transplantation has been performed properly in a case of transplantation of, for example, skin or a subcutaneous tissue with blood flow. Also, in other surgeries, a device is desired that enables easily recognizing a blood flow condition of a tissue, in order to assess a status of a tissue in a target region of a surgery and to identify, for example, an organ to be removed due to poor blood flow or an area of the tissue with poor blood flow. For performing such measurement, there is a known technology of measuring tissue oxygen saturation by receiving transmitted light of infrared light or near-infrared light radiated to a tissue as disclosed, for example, in Patent Document 2.

### PRIOR ART DOCUMENTS

### Patent Documents

Patent Document 1: Japanese Patent No. 6453914
Patent Document 2: Japanese Unexamined Patent Application Publication No. H10-234737

### SUMMARY OF THE INVENTION

### Problems to be Solved by the Invention

The aforementioned technique disclosed in Patent Document 1 has a problem that it takes time to obtain an accurate measured value, and thus measurement of tissue oxygen saturation in real time is difficult. There also is a problem that measured values may vary depending on a type of sensor or a measurement method employed, and may be affected by factors, such as measurement environment, and thus objective evaluation of the measured values is difficult.

In the technique disclosed in Patent Document 2, although measurement of oxygen saturation in real time can be performed, a depth where a tissue to be measured is located is not taken into consideration. Specifically, depending on a region to be measured, there might be a certain thickness of other tissues, such as fat, which affect measured values between a measurement target tissue and a skin on which a sensor is arranged. Thus, for measurement of accurate oxygen saturation of the target tissue, it is required to perform measurement considering the thickness of other tissues located between the measurement target tissue and the skin, in other words, considering the depth at which the measurement target tissue is located. However, the technique disclosed in Patent Document 2 does not include such consideration, and thus it is difficult to perform accurate measurement depending on the region to be measured.

Also, in the technique disclosed in Patent Document 2, simultaneous measurement at two or more locations is not expected. Thus, it is difficult to determine a status of an entire measurement target region based on measurement of respective oxygen saturations of different portions.

One aspect of the present disclosure has an object to provide a biological information collection device that is able to collect information regarding accurate tissue oxygen saturation in real time by simple operations. Another object is to provide a biological information collection sensor unit, a biological information collection sensor, and a biological information processing unit for use in the biological information collection device.

### Means for Solving the Problems

In order to achieve the aforementioned object, the present application provides measures below.

A biological information collection sensor unit, which is one aspect of the present disclosure, comprises two or more sensors, the sensors each comprising: a light emitter to emit near-infrared light or infrared light; and a light receiver to collect transmitted light including at least light that has transmitted through a measurement target tissue, and the sensors being arranged on surfaces of a measurement target region and configured to collect optical information regarding tissues of the measurement target region; and a processor configured to calculate tissue oxygen saturation information regarding oxygen saturation of the tissues based on the optical information, wherein the two or more sensors are sensors to be arranged in respective different areas of the measurement target region, and wherein the processor comprises an arithmetic processor configured to calculate the tissue oxygen saturation information for the respective sensors; and a notifier configured to output a notification signal when the number of the sensors that have collected the optical information used to calculate the tissue oxygen saturation information equal to or greater than a preset threshold satisfies a specified condition.

In the biological information collection sensor unit configured as above, the two or more sensors are arranged in respective different areas of the measurement target region, and the arithmetic processor calculates the tissue oxygen saturation information of the respective areas. Thus, the status of tissue oxygen saturation in the respective areas of the measurement target region can be recognized at the same time. Also, with this configuration, a notification signal is output from the notifier when the number of the sensors that have collected the optical information used to calculate the tissue oxygen saturation information equal to or greater than a preset threshold satisfies a specified condition. For example, a notification signal is output from the notifier when the number of the sensors that have collected the optical information used to calculate the tissue oxygen saturation information equal to or greater than the preset threshold is a specified number or more. Alternatively, a notification signal is output from the notifier when the number of the sensors that have collected the optical information used to calculate the tissue oxygen saturation information equal to or greater than the preset threshold is a specified number or less. Accordingly, it is possible to easily recognize a change in oxygenation state of a specified area of the measurement target region.

In one aspect of the present disclosure, the sensors preferably comprise: a first sensor configured to collect the optical information of a first area of the measurement target region; a second sensor configured to collect the optical information of a second area, which is different from the first area, of the measurement target region; and a third sensor configured to collect the optical information of a third area, which is different from the first area and the second area, of the measurement target region.

With the biological information collection sensor unit configured as above, information regarding tissue oxygen saturations in different three areas of the measurement target region is collected at the same time. Thus, the status of tissue oxygen saturation in the respective areas of the measurement target region can be recognized at the same time.

In one aspect of the present disclosure, the notifier preferably outputs the notification signal when the number of the sensors that have collected the optical information used to calculate the tissue oxygen saturation information equal to or greater than the threshold is two or more.

With the biological information collection sensor unit configured as above, when the tissue oxygen saturation information of at least two areas is equal to or greater than the threshold, a notification signal to notify that is output. Thus, it is possible to easily recognize that the number of areas each having a tissue oxygen saturation equal to or greater than the threshold is two or more.

In one aspect of the present disclosure, it is preferred that the measurement target region is a lower limb of a patient, the first sensor is a foot dorsum sensor to be arranged on a foot dorsum area, the second sensor is a foot bottom sensor to be arranged on a foot bottom area, and the third sensor is a lateral malleolus sensor to be arranged on a lateral malleolus area.

With the biological information collection sensor unit configured as above, tissue oxygen saturation information of specified portions of a foot, which is a distal part of a lower limb of a patient, is respectively collected. It is generally known that blood is perfused in respective portions of a foot by different blood vessels. Specifically, it is possible to obtain information regarding blood flow conditions of the blood vessels that perfuse blood in the respective portions from the tissue oxygen saturation information of the respective portions.

In one aspect of the present disclosure, it is preferred that the measurement target region is a lower limb of a patient, the first area is a portion in which blood is perfused by an anterior tibial artery, the second area is a portion in which blood is perfused by a posterior tibial artery, and the third area is a portion in which blood is perfused by a peroneal artery.

With the biological information collection sensor unit configured as above, the tissue oxygen saturation information of the respective portions of the patient's foot in which blood is perfused by different blood vessels is collected. Thus, it is possible to obtain information regarding blood flow conditions of the blood vessels that perfuse blood in the respective portions from the collected tissue oxygen saturation information of the respective portions.

In one aspect of the present disclosure, it is preferred that the biological information collection sensor unit is a sensor unit to be used to collect the tissue oxygen saturation information of the lower limb during revascularization of the lower limb, and the notifier is configured to output information regarding prognosis of the patient after the revascularization based on the number of the sensors that have collected the optical information used to calculate the tissue oxygen saturation information equal to or greater than the threshold.

With the biological information collection sensor unit configured as above, information regarding prognosis of the patient after the revascularization is output based on the number of the sensors that have collected the optical information used to calculate the tissue oxygen saturation information equal to or greater than the threshold. For example, information regarding an estimated cure rate of an ischemic ulcer or a wound after revascularization and an estimated lower limb amputation rate is output. Thus, it is possible to confirm treatment effects and prognosis of revascularization of a lower limb. The “estimated lower limb amputation rate” here means an estimated rate that sufficient treatment effects will not be obtained after revascularization of a lower limb, thus resulting in an amputation procedure of the lower limb as a treatment target.

A biological information collection device in another aspect of the present disclosure preferably comprises the aforementioned biological information collection sensor unit and a display configured to display the tissue oxygen saturation information.

With the biological information collection device configured as above, tissue oxygen saturation information collected and calculated by the biological information collection sensor unit is displayed on the display.

A biological information collection sensor unit in yet another aspect of the present disclosure is a biological information collection sensor unit that comprises a sensor arranged on a surface of a measurement target and configured to collect information regarding light that has transmitted through a tissue of the measurement target, and a processing unit configured to calculate tissue oxygen saturation information regarding oxygen saturation of the tissue based on the information collected by the sensor. The sensor is configured to collect transmitted light including at least light that has transmitted through a measurement target tissue located in an area separated from the surface of the measurement target on which the sensor is arranged by a preset depth distance or more, and comprises at least a light emitter to emit near-infrared light or infrared light and a light receiver to receive the transmitted light and output optical information regarding the transmitted light to the processing unit. The light receiver is configured to output identification information of the sensor linked to the depth distance, along with the optical information, and the processing unit comprises an arithmetic processor configured to calculate the tissue oxygen saturation information based on the optical information and the identification information.

With the biological information collection sensor unit configured as above, the processing unit calculates information regarding oxygen saturation of the measurement target tissue based on the optical information of the measurement target tissue collected by the sensor and on the identification information of the sensor that has collected the optical information.

It is preferred that the biological information collection sensor unit in yet another aspect of the present disclosure comprises two or more sensors with specified individual depth distances, and the arithmetic processor calculates the tissue oxygen saturation information for the respective sensors based on the optical information that is output by the respective sensors and the identification information.

With the biological information collection sensor unit configured as above, the processing unit calculates information regarding oxygen saturation of tissues for the respective sensors based on the optical information collected by the respective sensors and on the identification information of the respective sensors.

It is preferred that the processing unit in another aspect of the present disclosure comprises an outputter configured to output the tissue oxygen saturation information calculated by the arithmetic processor, and the outputter outputs the tissue oxygen saturation information calculated for the respective sensors as mutually identifiable information.

With the biological information collection sensor unit configured as above, tissue oxygen saturation information calculated for the respective sensors is output as mutually identifiable information.

It is preferred that the processing unit in another aspect of the present disclosure comprises a storage to store the identification information and at least information regarding the depth distance that are linked to each other, and that the arithmetic processor refers to the storage based on the identification information, obtains information regarding the corresponding depth distance, and calculates the tissue oxygen saturation information.

With the biological information collection sensor unit configured as above, the processing unit refers to the storage based on the identification information of the sensor and obtains information regarding the depth distance necessary to calculate tissue oxygen saturation information, and then calculates tissue oxygen saturation information.

It is preferred that a biological information collection device in still another aspect of the present disclosure comprises a biological information collection sensor unit and a display configured to display the tissue oxygen saturation information based on the information that is output by the outputter.

With the biological information collection device configured as above, tissue oxygen saturation information collected and calculated by the biological information collection sensor unit is displayed on the display.

### Effects of the Invention

With the biological information collection sensor unit of the present disclosure, it is possible to obtain information regarding the status of tissue oxygen saturation and blood flow conditions of respective areas of a measurement target region at the same time. Also, it is possible to easily recognize changes in oxygenation state and blood flow conditions of the measurement target region. Further, it is possible to obtain tissue oxygen saturation information depending on the depth distance set for the sensor in real time by a simple operation of only connecting the sensor to the processing unit.

### BRIEF DESCRIPTION OF THE DRAWINGS

FIG. 1 is a view illustrating a configuration of a biological information collection device in a first embodiment.

FIG. 2A is a view of a surface opposite to an attachment surface of a sensor of a biological information collection sensor unit in the first embodiment.

FIG. 2B is a side view of the sensor of the biological information collection sensor unit in the first embodiment.

FIG. 2C is a view of a reverse surface of the sensor, which is a surface on a side of the attachment surface, of the biological information collection sensor unit in the first embodiment.

FIG. 3A is a view showing a side surface on a connector side of a processing unit of the biological information collection sensor unit in the first embodiment.

FIG. 3B is a view showing a front surface of the processing unit of the biological information collection sensor unit in the first embodiment.

FIG. 3C is a view showing a side surface of the processing unit of the biological information collection sensor unit in the first embodiment.

FIG. 3D is a view showing a reverse surface of the processing unit of the biological information collection sensor unit in the first embodiment.

FIG. 4 is a block diagram illustrating the biological information collection device in the first embodiment.

FIG. 5 is a view illustrating a state where the sensors of the biological information collection sensor unit in the first embodiment are attached to a patient.

FIG. 6A is a view exemplifying information stored by a storage of the processing unit in the first embodiment.

FIG. 6B is a flowchart illustrating a process performed by the processing unit in the first embodiment.

FIG. 7 is a view illustrating a display example by a display device that configures the biological information collection device in the first embodiment.

FIG. 8 is a view illustrating a state of use of the biological information collection device in the first embodiment.

FIG. 9A is a view illustrating another state of use of the biological information collection device.

FIG. 9B is a view illustrating a further state of use of the biological information collection device.

FIG. 10 is a block diagram illustrating a biological information collection device in a second embodiment.

FIG. 11 is a view illustrating a running state of blood vessels in a lower limb of a patient.

FIG. 12A is a view illustrating an angiosome of an anterior tibial artery, specifically an area where blood perfusion is performed by the anterior tibial artery.

FIG. 12B is a view illustrating an angiosome of a posterior tibial artery, specifically an area where blood perfusion is performed by the posterior tibial artery.

FIG. 12C is a view illustrating an angiosome of a peroneal artery, specifically an area where blood perfusion is performed by the peroneal artery.

FIG. 13 is a view illustrating a state of use of the biological information collection device in the second embodiment.

FIG. 14 is a diagram illustrating results of a clinical trial performed using the biological information collection device in the second embodiment.

FIG. 15 is a diagram illustrating results of the clinical trial performed using the biological information collection device in the second embodiment.

### EXPLANATION OF REFERENCE NUMERALS

1, 1A . . . biological information collection device; 2, 2A . . . biological information collection sensor unit; 6 . . . display device; 20, 20A, 20B, 20C . . . sensor; 21 . . . light emitter (L.E.); 22, 22a, 22b, 22c . . . light receiver (L.R.); 23 . . . cable; 24 . . . circuit board; 25 . . . attachment surface; 26 . . . light shielding member; 27 . . . connector; 30 . . . processing unit; 31 . . . power button; 32 . . . state indicator; 33 . . . lid; 35a, 35b, 35c . . . connector; 41a, 41b, 41c . . . communication controller (COMM. CONT.); 42 . . . light emission controller (L.E. CONT.); 43 . . . insulation circuit (INSU. CIR.); 44 . . . ON/OFF circuit (ON/OFF CIR.); 45 . . . battery circuit (BAT. CIR.); 50 . . . control IC; 51 . . . communicator (COMM); 52 . . . antenna; 53 . . . arithmetic processor (ARITH. PROC.); 54 . . . storage; 55 . . . notifier; 61 . . . communicator (COMM); 62 . . . processor (PROC.); 63 . . . display storage (DISP. STORAGE); 64 . . . display/operation section (DISP./OPER.); AA, BB, CC . . . optical information; AA1, BB1, CC1 . . . tissue oxygen saturation information

### MODE FOR CARRYING OUT THE INVENTION

### First Embodiment

1. Description of Configuration

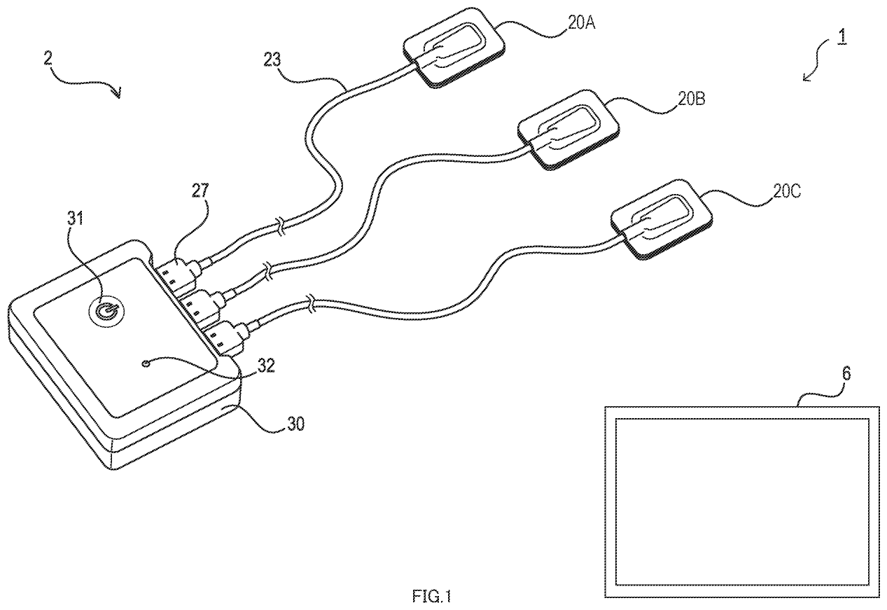

First, a description will be given of a configuration of a biological information collection device 1 in a first embodiment according to a technology of the present disclosure with reference to FIG. 1 to FIG. 4. In the present embodiment, a case is described in which the biological information collection device 1 according to the technology of the present disclosure is used as a medical device to measure information regarding oxygen saturation of a tissue of a patient, for example, for diagnosis purposes. This purpose of use of the biological information collection device 1 is an example and is not limitative. For example, the biological information collection device 1 may be used as a physicochemical device for measurement of biotissues of human bodies or animals, for example, for research purposes, or may be used as a device for measurement of biotissues for other purposes.

The biological information collection device 1 of the present embodiment comprises a biological information collection sensor unit 2 and a display device 6. As shown in FIG. 1, the biological information collection sensor unit 2 comprises sensors 20A, 20B, 20C and a processing unit 30. The biological information collection sensor unit 2 collects, from the sensors 20A, 20B, 20C arranged in a region of interest that is a region where measurement on a patient is performed, biological information of a tissue in the region of interest. The biological information collection sensor unit 2 also calculates information regarding tissue oxygen saturation based on collected information, and outputs a calculation result. Hereinafter, the information regarding tissue oxygen saturation calculated by the biological information collection sensor unit 2 is also referred to as “tissue oxygen saturation information”. Also, the region where measurement on a patient is performed is also referred to as a “region of interest”.

In the present embodiment, the description hereinafter is provided, employing an example in which the biological information collection sensor unit 2 calculates oxygen saturation (rSO2) of a tissue and total hemoglobin index (T-HbI) as tissue oxygen saturation information. The biological information collection sensor unit 2 may output other biological information as tissue oxygen saturation information, and tissue oxygen saturation information is not specifically limited to the aforementioned information.

Each of the sensors 20A, 20B, 20C is an optical sensor to be arranged on a surface of a region as a measurement target, such as a patient's skin, to collect biological information of the patient's tissue located in an area separated from the surface by a specified distance or more. In the description hereinafter, the specified distance from the surface is also referred to as a “depth distance”. Also, the patient's tissue located in the area separated from the surface is also referred to as a “measurement target tissue”. Further, the sensors 20A, 20B, 20C are also collectively referred to as “sensors 20”. Since the sensors 20A, 20B, 20C are the same except for differences in arrangement positions of some components, the description hereinafter will be provided using the same reference numeral for the same components. Arranging of the sensor 20, for example, on the patient's skin is also referred to as “attaching”.

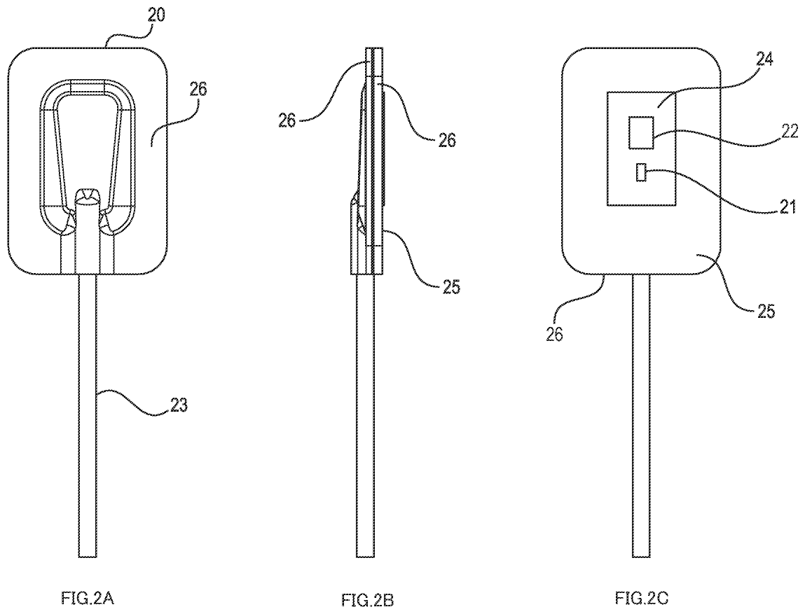

As shown in FIG. 2A, FIG. 2B, and FIG. 2C, the sensor 20 is a biological information collection sensor having a generally rectangular plate shape, in which both surfaces of a not-shown flexible circuit board are each covered with a light shielding member 26. In a substantially central portion of an attachment surface 25 of the sensor 20 to face the patient during use, a light emitter 21 and a light receiver 22 are arranged side by side on the circuit board. The light emitter 21 and the light receiver 22 are each electrically connected and fixed to the circuit board by a method, such as soldering. A central portion of the light shielding member 26 on the attachment surface 25 side is cut out substantially rectangularly, and a transparent cover 24 allowing transmission of light used for measurement is provided in the cut out area. As a material for the cover 24, any material may be employed which allows transmission of light used for measurement to a degree of not interrupting measurement. A cable 23 extends from the sensor 20 along a direction of alignment of the light emitter 21 and the light receiver 22. The cable 23 may extend in a direction different from the aforementioned. The cable 23 is a cable to electrically connect the sensor 20 and the processing unit 30. A connector 27 is provided at an end of the cable 23 opposite to the sensor 20. The connector 27 is to be connected to the processing unit 30.

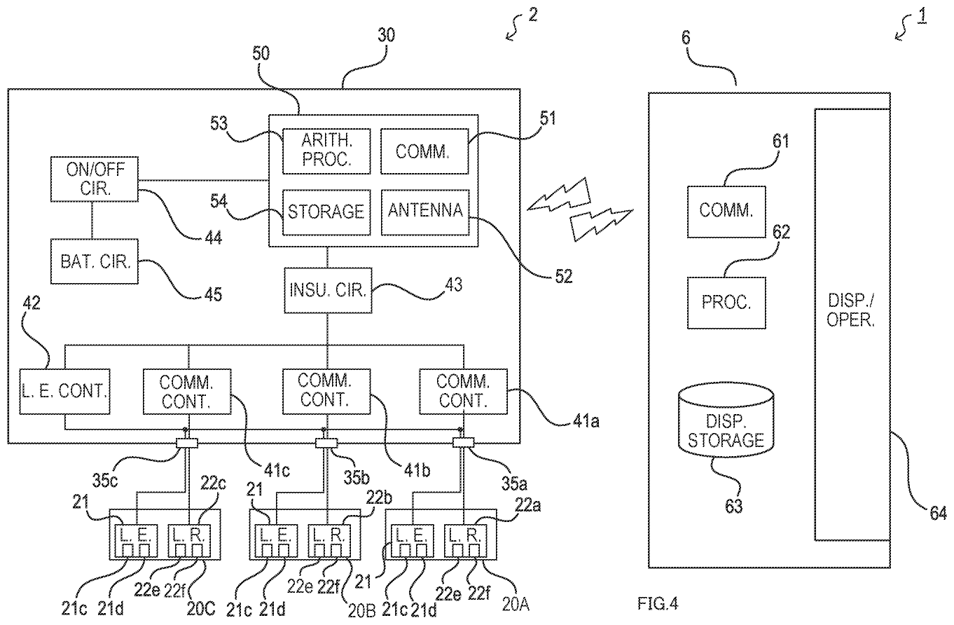

The light emitter 21 emits infrared light or near infrared light, and radiates the emitted light toward the measurement target tissue. The light emitter 21 comprises two light emission diodes that emit lights with respective different wavelengths. Hereinafter, the light emission diode is also referred to as an “LED”. In the present embodiment, as shown in FIG. 4, the light emitter 21 comprises an LED 21c to emit light with a wavelength of approximately 770 nm and an LED 21d to emit light with a wavelength of approximately 830 nm. The wavelengths of lights emitted by the LED 21c and the LED 21d may be different from the aforementioned. Also, the light emitter 21 may comprise a different type of light emitting element that emits light with a specified wavelength in place of the LED.

The light receiver 22 outputs received light in the form of a signal processable by the processing unit 30. In the description hereinafter, as shown in FIG. 4, the light receiver 22 of the sensor 20A is also referred to as a light receiver 22a. The light receiver 22 of the sensor 20B is also referred to as a light receiver 22b. The light receiver 22 of the sensor 20C is also referred to as a light receiver 22c.

The light receiver 22 comprises two photodiodes that each output an electric signal corresponding to the received light. In the description hereinafter, a photodiode is also referred to as a “PD”. Also, the two photodiodes are also referred to as PD 22e and PD 22f, as shown in FIG. 4. The light receiver 22 has a function of outputting an electric signal output from the PD as a signal processable by the processing unit 30, and a function of storing identification information of the sensor 20, which will be detailed below. In the description hereinafter, information regarding the received light that the light receiver 22 outputs is also referred to as “optical information”. In the present embodiment, the light receiver 22 performs A/D conversion of the signal based on the received light, which is output by the PD 22e and the PD 22f, and then outputs the converted signal. The light receiver 22 may comprise another type of light receiving element to output an electric signal corresponding to the received light, in place of the PD.

The sensor 20 radiates infrared light or near infrared light emitted by the light emitter 21 from a skin surface where the sensor 20 is arranged toward a measurement target tissue. The sensor 20 also receives transmitted light including at least light that has transmitted through the measurement target tissue, and outputs optical information based on the transmitted light to the processing unit 30. In the present embodiment, a description will be given of a case in which respective different depth distances are previously specified for the sensors 20A, 20B, 20C.

Specifically, in the present embodiment, it is specified that the sensor 20A has a depth distance of approximately 2 mm, the sensor 20B has a depth distance of approximately 4 mm, and the sensor 20C has a depth distance of approximately 8 mm. More specifically, the sensor 20A is configured to collect transmitted light including at least light that has transmitted through the measurement target tissue located in an area spaced approximately 2 mm or more apart from the skin surface where the sensor 20A is arranged. The sensor 20B is configured to collect transmitted light including at least light that has transmitted through the measurement target tissue located in an area spaced approximately 4 mm or more apart from the skin surface where the sensor 20B is arranged. The sensor 20C is configured to collect transmitted light including at least light that has transmitted through the measurement target tissue located in an area spaced approximately 8 mm or more apart from the skin surface where the sensor 20C is arranged. Hereinafter, the depth distance is also referred to as a “depth” or a “measurement depth”.

The depth distance of the sensor 20 in the present embodiment is specified based on a distance between the light emitter 21 and the light receiver 22. The depth distance of the sensor 20 may be specified based on, for example, other parameters. Also, the depth distance of the sensor 20 may be different from the aforementioned. Further, the same depth distance may be specified for each of the sensors 20A, 20B, 20C.

The light receiver 22 has a function of storing identification information linked to the depth distance of the sensor 20. The light receiver 22 outputs stored identification information as well as optical information as output information to the processing unit 30. In the present embodiment, the light receiver 22a, the light receiver 22b, and the light receiver 22c store identification information linked to the previously specified depth distances of the sensors 20A, 20B, 20C, respectively. Specifically, the light receiver 22a stores identification information a02, the light receiver 22b stores identification information b04, and the light receiver 22c stores identification information c08. The sensor 20 has a form suitable for being applied and attached to a surface of a skin or the like, as shown in FIG. 2. This form of the sensor 20 is illustrative only, and the form as shown is not limitative. As the form of the sensor 20, any form may be employed that is suitable for contacting various regions and collecting information regarding oxygen saturation. For example, the sensor 20 may be in a form in which the light emitter 21, the light receiver 22, and other components are arranged in a finger sack shaped support. Alternatively, the sensor 20 may be configured such that the light shielding member 26 on the attachment surface 25 side has a greater size to achieve an increased attachment performance when attached to a patient. That is, the sensor 20 may be in various forms depending on intended purposes and measurement target regions. Also, for a material forming an exterior of the sensor 20, various materials may be used that are suitable to contact various regions and collect information regarding oxygen saturation.

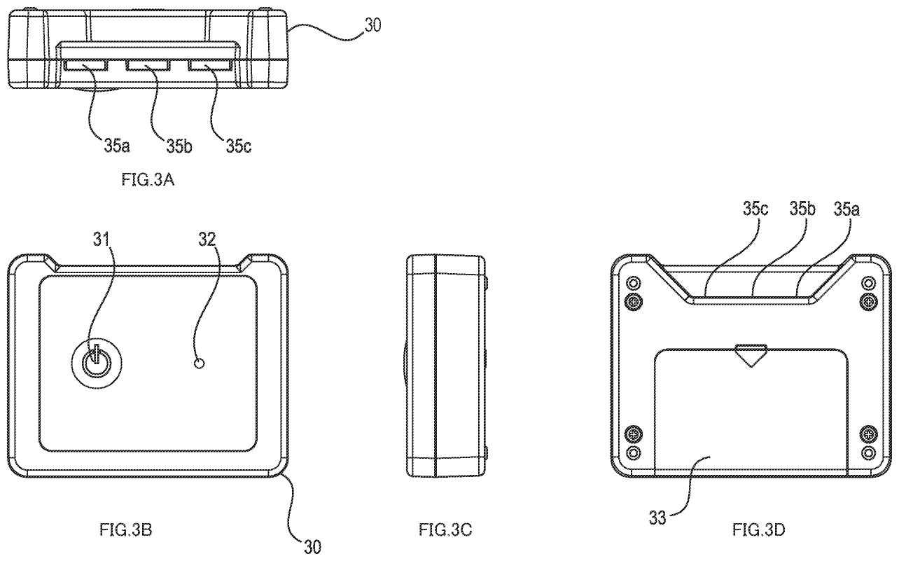

The processing unit 30 is a biological information processing unit to calculate information regarding oxygen saturation of respective measurement target tissues based on output information that is output by the sensor 20. The processing unit 30 has a generally rectangular box shape and includes a front surface provided with a power button 31 and a state indicator 32. In a side surface of the processing unit 30, connectors 35a, 35b, 35c into each of which the connector 27 is inserted for connection are provided as shown in FIG. 3A and FIG. 3B. The connectors 35a, 35b, 35c are the same connectors, and thus the connector 27 of the sensor 20 may be inserted into any one of the connectors 35a, 35b, 35c for connection. In the description hereinafter, the connectors 35a. 35b, 35c are also collectively referred to as “connectors 35”.

A lid 33 is provided in a reverse surface of the processing unit 30. The lid 33 is used, for example, for replacing a battery as a power source of the processing unit 30, and is configured to be freely attachable and detachable.

As shown in FIG. 4, the processing unit 30 comprises communication controllers 41a, 41b, 41c, a light emission controller 42, an insulation circuit 43, an ON/OFF circuit 44, a battery circuit 45, and a control IC 50. The communication controllers 41a, 41b, 41c are each a communication driver to perform communication with the light receiver 22. The communication controllers 41a, 41b, 41c are provided corresponding to the connector 35a, 35b, 35c, respectively. Hereinafter, the communication controllers 41a, 41b, 41c are also collectively referred to as “communication controllers 41”. Also, a set of one connector 35 and one communication controller 41 corresponding to the connector 35 is also referred to as a “channel”. Further, a set of the connector 35a and the communication controller 41a is also referred to as a “channel CH”. Similarly, a set of the connector 35b and the communication controller 41b is also referred to as a “channel CH2”, and a set of the connector 35c and the communication controller 41c is also referred to as a “channel CH3”.

The light emission controller 42 controls radiation of light by the light emitter 21. Specifically, the light emission controller 42 outputs and stops electric current required for light emission of the LED 21c and the LED 21d of the light emitter 21 in accordance with a signal from the control IC 50, which will be detailed below, to thereby control radiation of light by the light emitter 21. In the present embodiment, measurement is performed by repeatedly collecting optical information in the order of the channel CH1, the channel CH2. and the channel CH3 in accordance with a control signal from the control IC 50. Thus, the LED 21c and the LED 21d of each of the sensors 20A, 20B, 20C are repeatedly turned on and off sequentially in accordance with the signal from the control IC 50. Control of the channel CH1, the channel CH2, and the channel CH3 may be performed in a different order from the aforementioned. Also, the light emitter 21 and the light receiver 22 may be controlled by any other control method that allows obtainment of optical information.

The insulation circuit 43 is an insulation circuit element to ensure insulation between a circuit on a side of the battery circuit 45 and a circuit on a side of the communication controller 41. The ON/OFF circuit 44 supplies and stops power from the battery circuit 45 in accordance with an action of the power button 31. When the ON/OFF circuit 44 is activated and power is supplied, the state indicator 32 is turned on to indicate that power is supplied. The battery circuit 45 is a power source of the processing unit 30 and comprises a booster circuit in addition to a battery.

The control IC 50 has a function of controlling the processing unit 30 as well as a function of wirelessly communicating with the device 6, which will be detailed below. The control IC 50 comprises a communicator 51, an antenna 52, an arithmetic processor 53, and a storage 54.

The communicator 51 performs wireless communication with a communicator 61 of the display device 6 through the antenna 52. In the present embodiment, a description hereinafter will be made by using an example of performing communication between the communicator 51 and the communicator 61 in accordance with the BLE protocol (Bluetooth Low Energy), which is an international wireless communication protocol. As used herein, “Bluetooth” is a registered mark. The communicator 51 may perform communication with the communicator 61 in accordance with another wireless protocol. The communicator 51 and the antenna 52 in the present embodiment correspond to an example of an outputter.

The arithmetic processor 53 calculates a tissue oxygen saturation (rSO2) and a total hemoglobin index (T-HbI) of a measurement target tissue based on output information from the light receiver 22. The calculated tissue oxygen saturation (rSO2) and total hemoglobin index (T-HbI) are linked to information related to a channel of input and information related to a time when optical information is obtained, and then are output by the communicator 51 and the antenna 52. Alternatively, the calculated tissue oxygen saturation (rSO2) and total hemoglobin index (T-HbI) may be further linked to information related to a time when the tissue oxygen saturation information is calculated, and then output by the communicator 51 and the antenna 52.

The arithmetic processor 53 also has a function of communicating with the light emission controller 42 and the light receiver 22, to thereby control collection of optical information by the sensor 20 and control communication with the display device 6 by the communicator 51.

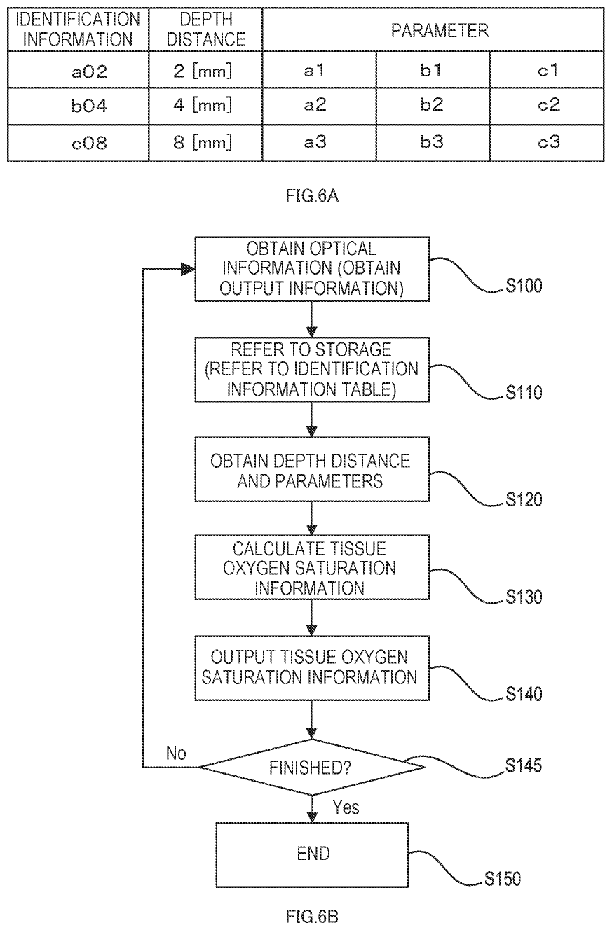

The storage 54 is a storage medium, such as a non-volatile semiconductor memory, to store parameters necessary to calculate tissue oxygen saturation information and other information. In the present embodiment, the storage 54 stores respective identification information of the sensors 20 and corresponding depth distances of the sensors 20 in a linked manner. Also, the storage 54 stores respective identification information of the sensors 20 and parameters necessary to calculate tissue oxygen saturation information based on optical information collected by the corresponding sensors 20 in a linked manner. In the present embodiment, the storage 54 stores an identification information table recording the depth distances of the sensors 20 and the parameters necessary to calculate tissue oxygen saturation information linked to the identification information. More specifically with reference to FIG. 6A, the identification information table records a depth distance of “2 mm” and parameters of “a1, b1, c1” necessary to calculate tissue oxygen saturation information, which are linked to the identification information a02. Also, a depth distance of “4 mm” and parameters of “a2, b2, c2” necessary to calculate tissue oxygen saturation information are linked to the identification information b04 and recorded. Further, a depth distance of “8 mm” and parameters of “a3, b3, c3” necessary to calculate tissue oxygen saturation information are linked to the identification information c08 and recorded. The depth distance and the parameters necessary to calculate tissue oxygen saturation information in the present embodiment correspond to one example of information related to depth distance.

The display device 6 displays the tissue oxygen saturation and the total hemoglobin index calculated by the arithmetic processor 53 in the form of a graph or values. In the present embodiment, the display device 6 is a tablet PC that is a portable information processor. The display device 6 may be another type of laptop PC or another information processor provided with a display device.

The display device 6 comprises the communicator 61, a processor 62, a display storage 63, and a display/operation section 64. The communicator 61 performs communication with the communicator 51 of the processing unit 30. The communicator 61 has a function of receiving information, such as tissue oxygen saturation and total hemoglobin index, from the communicator 51. The communicator 61 also has a function of sending, for example, information regarding an operation that is input from the display/operation section 64 to the communicator 51.

The processor 62 performs processing necessary to display the tissue oxygen saturation information received by the communicator 61 on the display/operation section 64. The processor 62 also performs processing of information regarding an operation that is input from the display/operation section 64. The display storage 63 is a storage medium to store the received tissue oxygen saturation information.

The display/operation section 64 is a touch panel display that is a display to display information, such as tissue oxygen saturation information, and also has a function of an input device to receive an operation by a user. The display/operation section 64 may be configured with a display device, such as a monitor, and with an input device, such as a mouse and a keyboard. The display device 6 or the display/operation section 64 in the present embodiment corresponds to one example of a display.

2. Description of Operation

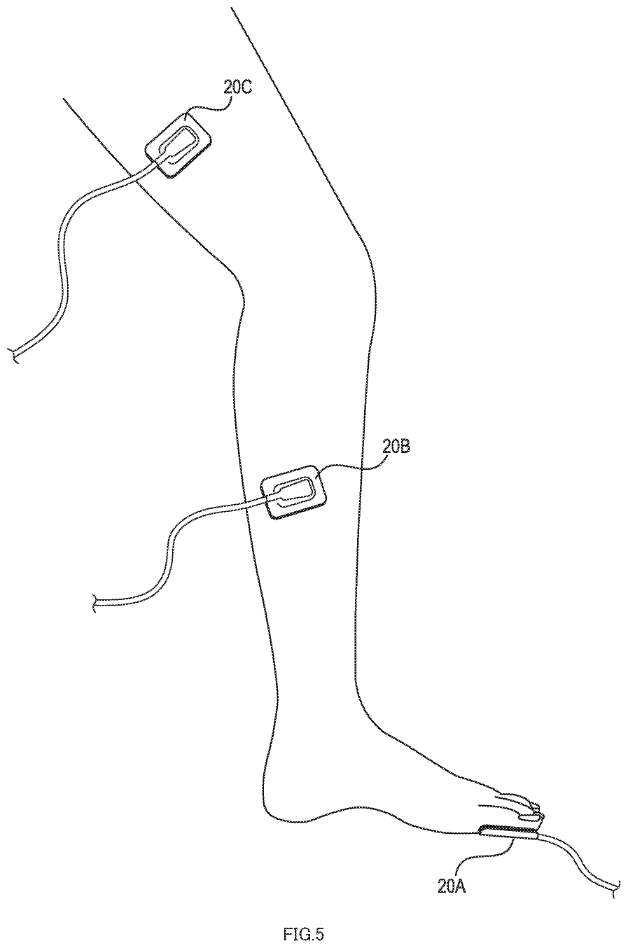

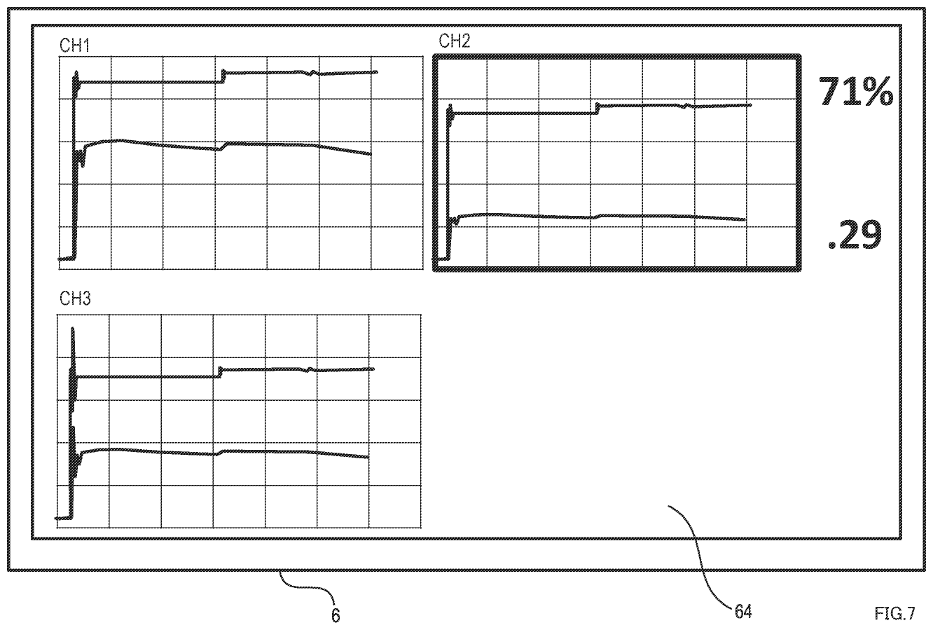

Next, a description will be given of an operation of the biological information collection device 1 in accordance with its method of use with reference to FIG. 5, FIG. 6, and FIG. 7. In the present embodiment, the description will be given of an example where the biological information collection device 1 is used for a test to diagnose blood flow conditions of a lower limb of a patient.

First, the connectors 27 of the sensors 20A, 20B, 20C are connected to the respective connectors 35 of the processing unit 30. In the present embodiment, a description hereinafter will be given of an example where the connector 27 of the sensor 20A is connected to the connector 35a, the connector 27 of the sensor 20B is connected to the connector 35b, and the connector 27 of the sensor 20C is connected to the connector 35c. The connector 27 of each sensor 20 may be connected to any connector 35 different from the aforementioned.

Subsequently, the sensors 20A, 20B, 20C are each arranged and fixed on a skin of a region in which measurement is performed. In this case, the sensor 20 is arranged to cause the attachment surface 25 to contact the skin of the patient. In the present embodiment, as shown in FIG. 5, in light of thicknesses of tissues not subject to measurement, such as subcutaneous fat and bones, in the measurement target region, the sensor 20C having a long depth distance is arranged on the patient's thigh, and the sensor 20A having a short depth distance is arranged on a peripheral part. Then, the sensor 20B is arranged between the sensor 20A and the sensor 20C. After the sensors 20 are arranged in respective positions, the sensors 20 are fixed on the patient using, for example, a medical tape. An adhesive member may be applied to the attachment surface 25, to thereby fix the sensors 20 on the patient. The sensors 20A, 20B, 20C having the same depth distance may be employed.

Subsequently, the power button 31 is operated to turn on power of the processing unit 30. When power of the processing unit 30 is turned on, power is also supplied to the sensor 20, which is enabled to perform measurement and starts collecting optical information.

More specifically, the light emitters 21 emit light according to a control signal from the arithmetic processor 53. Specifically, electricity required for light emission is supplied to the LEDs 21c and the LEDs 21d of the respective light emitters 21 according to the control signal, and then infrared lights or near infrared lights with different wavelengths are radiated to the patient's skin. Also, the light receivers 22 each start a process of receiving transmitted light and outputting output information according to the control signal from the arithmetic processor 53.

Specifically, the PDs 22e and the PDs 22f of the respective light receivers 22 output signals according to strengths of respective received transmitted lights, and the light receivers 22 output optical information resulting from processing of the signals. In this case, the light receivers 22 each output identification information of the sensor 20 along with the optical information.

Hereinafter, the optical information that is output by the light receivers 22a, 22b, 22c of the sensors 20A, 20B, 20C will be described specifically as optical information AA, BB, CC, respectively. Specifically, the light receiver 22a links, as output information, the optical information AA to the identification information a02, and outputs the same as output information. Also, the light receiver 22b links the optical information BB to the identification information b04, and outputs the same as output information. Further, the light receiver 22c links the optical information CC to the identification information c08, and outputs the same as output information.

The output information that is output from the light receivers 22 is input to the arithmetic processor 53 of the control IC 50 through the communication controllers 41 corresponding to the connectors 35 to which the respective sensors 20 are connected.

The arithmetic processor 53 performs arithmetic processing of each output information that is input from the communication controller 41, and calculates tissue oxygen saturation information based on the optical information and the identification information included in the output information. In the present embodiment, a description will be given of an example where the arithmetic processor 53 calculates tissue oxygen saturation information of the measurement target tissue by calculation means using the spatially resolved method. In the calculation means using the spatially resolved method, the arithmetic processor 53 obtains spatial gradient in the spatially resolved method based on the optical information and information regarding respective distances between the LED 21c and the LED 21d of the light emitter 21, and the PD 22e and the PD 22f of the light receiver 22. Then, the arithmetic processor 53 calculates information regarding tissue oxygen saturation using a parameter defined by the depth distance, specifically a coefficient. The arithmetic processor 53 may calculate the tissue oxygen saturation information using other calculation means.

Hereinafter, a specific description will be given of the processing performed by the arithmetic processor 53 taking the optical information AA as an example. First, the arithmetic processor 53 obtains output information of the sensor 20A from the communication controller 41a (S100). Then, the arithmetic processor 53 refers to the identification information table in the storage 54 based on the identification information a02 linked to the optical information AA, as exemplified in FIG. 6A (S110), and obtains a depth distance of “2 mm” and parameters “a1, b1, c1” linked to the identification information (S120). The arithmetic processor 53 may perform, in S120, a process of calculating parameters required for calculation of the tissue oxygen saturation information based on the depth distance obtained from the identification information table, thereby obtaining parameters “a1, b1, c1”.

Subsequently, the arithmetic processor 53 performs a calculation process to obtain tissue oxygen saturation information using the spatially resolved method based on the optical information AA, the depth distance of “2 mm”, and the parameters “a1, b1, c1” (S130). More specifically, the arithmetic processor 53 calculates, as tissue oxygen saturation information AA1, a tissue oxygen saturation and a total hemoglobin index of the measurement target tissue in a region where the sensor 20A is arranged.

Subsequently, the arithmetic processor 53 performs a process of outputting the calculated tissue oxygen saturation information AA1 to the communicator 51 (S140). In this case, the arithmetic processor 53 outputs the tissue oxygen saturation information AA1 linked to information regarding a time of its calculation. Also, the arithmetic processor 53 outputs the tissue oxygen saturation information AA1 linked to information to distinguish the tissue oxygen saturation information AA1 from tissue oxygen saturation information calculated based on other optical information BB, CC. In the present embodiment, the arithmetic processor 53 outputs information linked to information regarding a channel through which the optical information is input. Specifically, the arithmetic processor 53 performs a process of outputting information regarding the channel CH1 through which the optical information AA is input and time information regarding the time of calculation of the tissue oxygen saturation information AA1, linking these information to the tissue oxygen saturation information AA1. Similarly, the arithmetic processor 53 performs a process of outputting the calculated tissue oxygen saturation information BB1 to the communicator 51. In this case, the arithmetic processor 53 outputs the tissue oxygen saturation information BB1 linked to information to distinguish the tissue oxygen saturation information BB1 from other tissue oxygen saturation information. Further, the arithmetic processor 53 performs a process of outputting information regarding the channel CH2 through which optical information BB is input and time information regarding a time of calculation of the tissue oxygen saturation information BB1, linking these information to the tissue oxygen saturation information BB1. The arithmetic processor 53 also performs a process of outputting the calculated tissue oxygen saturation information CC1 to the communicator 51. In this case, the arithmetic processor 53 outputs the tissue oxygen saturation information CC1 linked to information to distinguish the tissue oxygen saturation information CC1 from other oxygen saturation information. The arithmetic processor 53 also performs a process of outputting the tissue oxygen saturation information CC1 linked to information regarding the channel CH3 through which the optical information CC is input and time information regarding a time of calculation of the tissue oxygen saturation information CC1.

The arithmetic processor 53 repeats the aforementioned processes until the user performs an operation to finish the measurement (“No” in S 145). When the user performs a process of finishing, such as operating the power button 31, (“Yes” in S145), the arithmetic processor 53 terminates the processing (S150).

When the arithmetic processor 53 performs a process of outputting the tissue oxygen saturation information AA1, BB1, CC1, the communicator 51 performs a process of sending the information as wireless signals through the antenna 52. Specifically, the arithmetic processor 53 performs a transmission process of outputting, as wireless signals, the input tissue oxygen saturation information AA1, BB1, CC1 linked to information regarding the respective channels CH1, CH2, CH3 and the time information. The communicator 51 performs the transmission process at specified time intervals. In the present embodiment, the time interval is set to 0.5 seconds. The time interval may be a time interval, such as a sampling rate employed in another general purpose biological information monitor. Alternatively, the time interval may be such a time interval that at least the user can determine that collection and display of information regarding the tissue oxygen saturation is displayed in real time.

When the communicator 51 performs the transmission process, the tissue oxygen saturation information AA1, BB1, CC1 and the respective information linked to the tissue oxygen saturation information AA1, BB1, CC1 is transmitted as wireless signals from the antenna 52. As exemplified in FIG. 7, the display device 6 receives the signals transmitted from the antenna 52, and performs a process of displaying the tissue oxygen saturation information of the respective channels on the display/operation section 64. Specifically, the processor 62 performs a process of processing the wireless signals received by the communicator 61, and displaying the tissue oxygen saturation information AA1, BB1. CC1 on the display/operation section 64. In the present embodiment, the processor 62 displays graphs showing temporal changes of the tissue oxygen saturation information AA1, BB1, CC1 at a transmission interval of the communicator 51. In the present embodiment, a description will be given of a case where the transmission interval is 0.5 seconds. The time interval of display may be, for example, any time interval between 0.1 and 1 second, or may be a time interval of 1 second or more. Also, the user may optionally select or change the time interval within a specified range. Further, display may be performed at a time interval different from the aforementioned if the time interval is such that the user can determine that the tissue oxygen saturation information is displayed in real time. The processor 62 may display the tissue oxygen saturation information by other display methods, and may perform the display at a time interval different from the transmission interval of the communicator 51. Also, the processor 62 may perform a process of switching a display screen of the tissue oxygen saturation information according to a user's operation performed on a not-shown operation screen displayed on the display/operation section 64.

Further, the processor 62 performs a process of linking the received tissue oxygen saturation information AA1. BB1, CC1 to the information regarding the respective channels and the time information, and storing these information in the display storage 63.

In the biological information collection device 1, measurement is made by the sensors 20 using electronic devices, such as LEDs and PDs, and the tissue oxygen saturation information is calculated. Thus, the tissue oxygen saturation information of the measurement target tissue can be measured in real time.

The arithmetic processor 53 in the processing unit 30 of the biological information collection sensor unit 2 calculates the tissue oxygen saturation information based on the optical information of the measurement target tissue collected by the sensors 20, and on the identification information of the sensors 20 that collected the optical information. Generally in a case where the tissue oxygen saturation information is measured by radiating infrared light or near infrared light, it is required to eliminate influences of subcutaneous fat, bones, and the like to collect more accurate information of the measurement target tissue. Specifically, a sensor is required to be set to collect optical information of a tissue located at a depth distance depending on a region to which the sensor is attached. When the depth distance is set to the sensor in such manner, at least the set depth distance is required to be input and set to each sensor each time tissue oxygen saturation information is calculated. However, in the biological information collection sensor unit 2 of the present embodiment, the light receiver 22 of the sensor 20 stores the identification information linked to the depth distance and outputs the identification information together with the optical information, and thus such individual setting is not required. Specifically, in the biological information collection sensor unit 2 of the present embodiment, a simple operation of only connecting any sensor 20 to the processing unit 30 enables calculation of tissue oxygen saturation information depending of the depth distance set to the sensor 20.

The biological information collection sensor unit 2 comprises two or more sensors 20, and the processing unit 30 calculates the tissue oxygen saturation information for the respective sensors 20 based on the optical information collected by the respective sensors 20 and the respective identification information. Thus, by a simple operation of only connecting the two or more sensors 20 to any of the connectors 35 of the processing unit 30 and arranging the sensors 20 in different areas, tissue oxygen saturation information of measurement target tissues of two or more areas can be obtained at the same time.

The processing unit 30 of the biological information collection sensor unit 2 comprises the communicator 51 and the antenna 52 that output the tissue oxygen saturation information calculated for the respective sensors 20 in such a manner that the respective channels are identifiable. Thus, the respective tissue oxygen saturation information based on the optical information obtained from the two or more sensors 20 can be output at the same time. Also, the calculated tissue oxygen saturation information is output according to the BLE standard, which is an international wireless communication standard. Accordingly, it is possible to enable information processors, such as commercially available tablet PCs, or other monitor devices to display tissue oxygen saturation information for the respective channels collected and calculated by the biological information collection sensor unit 2.

The processing unit 30 of the biological information collection sensor unit 2 comprises the storage 54 to store at least the identification information table recording the identification information of the sensors 20 and the information regarding respective depth distances that are linked to each other. Thus, the arithmetic processor 53 can obtain, for example, parameters necessary for calculating the tissue oxygen saturation information by simply referring to the storage 54 based on the identification information of the sensor 20, and can calculate the tissue oxygen saturation information through a simple arithmetic processing. Also, in a case of adding the sensor 20 with a new depth distance, it is sufficient to simply add information regarding the added sensor 20 to the identification information table, which facilitates addition of the sensor 20 of a new type.

The tissue oxygen saturation information collected and calculated by the biological information collection sensor unit 2 is displayed on the display device 6. In the present embodiment, the biological information collection sensor unit 2 and the display device 6 communicate wirelessly. Thus, it is possible to confirm information regarding oxygen saturation of a tissue measured at any location suitable for an intended purpose. For example, in a case of using the above-described biological information collection device 1 during a surgery, it is possible to move the display device 6 to a position for easy viewing by medical workers who conduct the surgery depending on conditions of the surgery. It is also possible to install the display device 6 in a remote place from the biological information collection sensor unit 2, to confirm information regarding the measured tissue oxygen saturation in a remote room from an operating room.

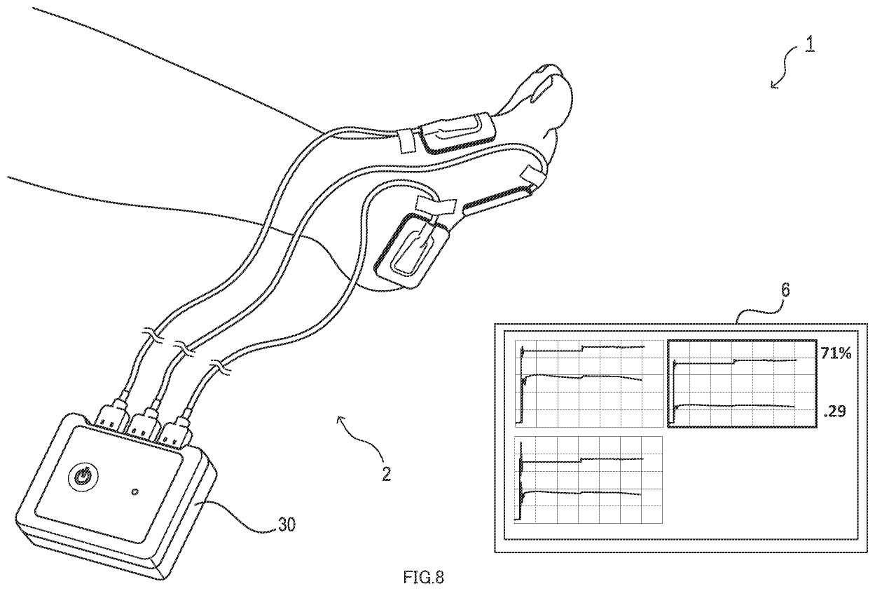

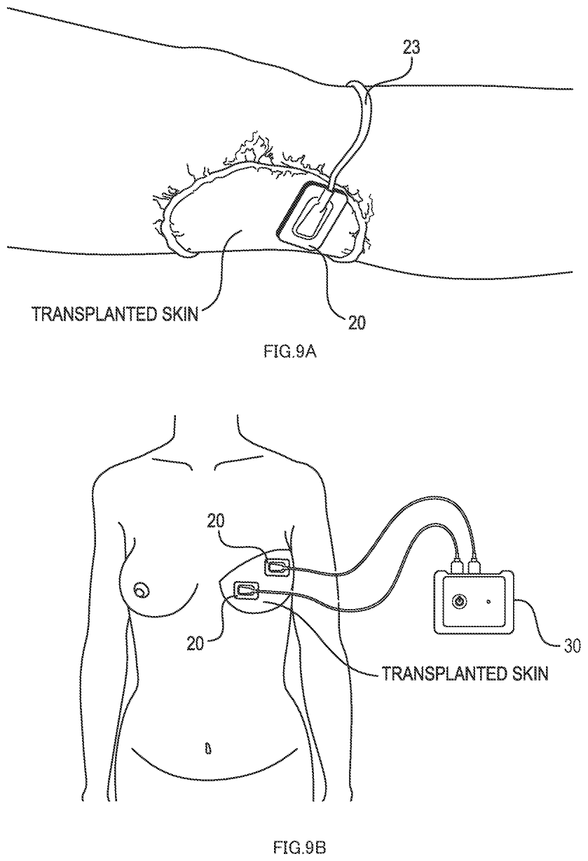

The technical scope of the present disclosure is not limited to the above-described embodiment, but may be modified in various forms within a scope not departing from the spirit of the technology. For example, while the aforementioned embodiment describes an example of collecting tissue oxygen saturation information of the thigh and distal parts in the lower limb of a patient, the biological information collection device 1 may be used for collecting tissue oxygen saturation information of tissues in the periphery of the lower limb, as shown in FIG. 8. Also, as shown in FIG. 9A and FIG. 9B, the biological information collection device 1 may be used for collecting tissue oxygen saturation information of a free flap that is a skin collected along with blood vessels from another region and transplanted by vascular anastomosis, for example, in breast reconstruction surgery or skin cancer surgery, or tissue oxygen saturation information of other regions. Further, the biological information collection device 1 may be used, for example, during surgery for collecting tissue oxygen saturation information of a tissue of an organ, such as stomach, intestinal tract, liver, kidney, or heart, by arranging the sensor 20 directly on a surface of the organ or the like. Moreover, the biological information collection device 1 may be used for collecting tissue oxygen saturation information of other measurement target regions.

In the aforementioned embodiment, the description has been given of a case where parameters necessary for calculating the tissue oxygen saturation information linked to the identification information are recorded in the identification information table of the storage 54. Alternatively, algorithms to be used for calculating the tissue oxygen saturation information linked to the identification information may be recorded in the identification information table. In this case, it is possible to calculate the tissue oxygen saturation information using an algorithm suitable for the depth distance depending on the connected sensor 20.

In the aforementioned embodiment, the description has been given of a case where the light receiver 22 stores the identification information of the sensor 20. However, another device and the like provided in the sensor 20 may store the information. For example, the identification information may be recorded by a DIP switch configured with two or more ON/OFF switches. In this case, it is possible to provide the sensor 20 with a simple configuration.

In the aforementioned embodiment, the description has been given of a configuration in which the biological information collection sensor unit 2 and the display device 6 communicate wirelessly. Alternatively, it may be configured such that the biological information collection sensor unit 2 and the display device 6 communicate by wire. This configuration can provide a simple and inexpensive biological information collection device 1. Also, for example, the processing unit 30 may comprise a display. This configuration can provide a biological information collection device with a further simple configuration

### Second Embodiment

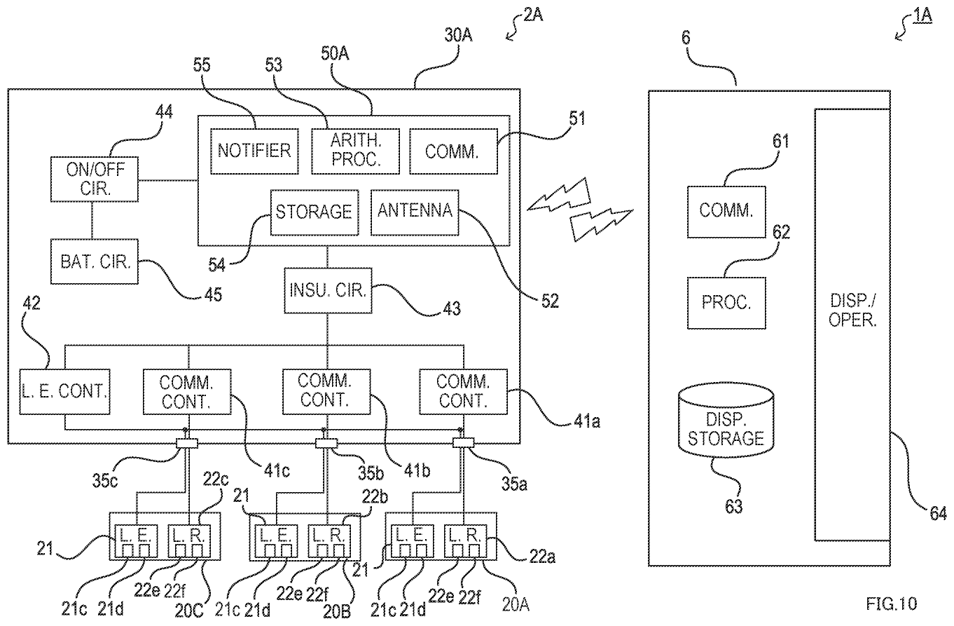

Subsequently, a description will be given of a biological information collection device 1A of a second embodiment according to the technology of the present disclosure. The biological information collection device 1A of the present embodiment has approximately the same configuration as in the first embodiment, as shown in FIG. 10, but is different from the first embodiment in that a processing unit 3A comprises a notifier 55. In the description hereinafter, the same components as in the first embodiment will be assigned the same reference numerals without further explanation, and only differences will be explained.

1. Description of Configuration

The biological information collection device 1A of the present embodiment comprises a biological information collection sensor unit 2A and the display device 6. The biological information collection sensor unit 2A comprises the sensors 20A, 20B, 20C and a processing unit 30A.

The processing unit 30A comprises the notifier 55. The notifier 55 has a function of comparing the respective tissue oxygen saturation information calculated by the arithmetic processor 53 for respective channels with a preset threshold. In the present embodiment, an example case will be described where the notifier 55 performs a process of sequentially comparing tissue oxygen saturation values included in the tissue oxygen saturation information calculated by the arithmetic processor 53 with the preset threshold at preset time intervals. The notifier 55 may perform a process of comparing other information included in the tissue oxygen saturation information with a corresponding threshold.

The notifier 55 also has a function of outputting a notification signal when the number of channels of the tissue oxygen saturation information each having a value equal to or greater than the threshold satisfies a specified condition. In other words, the notifier 55 has a function of outputting a notification signal to notify a user of the fact, if applicable, that the number of the sensors 20 that collected optical information used for calculating the tissue oxygen saturation information each having a value equal to or greater than the threshold satisfies a specified condition.

The notifier 55 further has a function of storing the threshold to be used when performing the notification. In the present embodiment, “50%” is set as the threshold of the tissue oxygen saturation for curing ulcers and wounds, and the notifier 55 stores this value. The notifier 55 may store different thresholds for the respective channels. Alternatively, the notifier 55 may store, as the threshold, a value that the user has input by operating, for example, the display/operation section 64. The value of the threshold is exemplary and is not limited to 50%. For example, in a case of using the biological information collection device 1A for a purpose other than curing ulcers or wounds, a threshold of the tissue oxygen saturation suitable for the purpose can be set, and different thresholds for the respective measurement target tissues may be set. The threshold may be a different value that is set based on results of actually performed treatment or on other knowledge. Also, a section different from the notifier 55 may store the threshold.

In the present embodiment, the notifier 55 is configured to output a notification signal to the display device 6, in a case where the number of channels of the tissue oxygen saturation information each having a value equal to or greater than the threshold is two or more. More specifically, the notifier 55 outputs a signal to notify, when the number of channels of the tissue oxygen saturation information each having a value equal to or greater than the threshold reaches two, that the number of channels each having a value equal to or greater than the threshold is two. Further, the notifier 55 outputs a signal to notify, when the number of channels of the tissue oxygen saturation information each having a value equal to or greater than the threshold reaches three, that the number of channels each having a value equal to or greater than the threshold is three. The notifier 55 may be configured to output signals sequentially notifying the number of channels of the tissue oxygen saturation information each having a value equal to or greater than the threshold.

2. Description of Operation

Next, a description will be given of an operation of the biological information collection device 1A according to its use method. In the description hereinafter, an actual case will be described in which the biological information collection device 1A is used for measurement of tissue oxygen saturation of a lower limb of a patient who undergoes revascularization for lower limb arteriosclerosis obliterans. Specifically, the description will be made regarding a case in which the biological information collection device 1A is used for measurement of tissue oxygen saturation information regarding a treatment target region of revascularization in a leg of the patient, in other words, a peripheral part of the lower limb. Since there is a close relationship between tissue oxygen saturation information and blood flow flowing in tissues, it is possible to recognize a state of blood flow in the lower limb, as a treatment target, by performing measurement of tissue oxygen saturation information using the biological information collection device 1A. In the present embodiment, the same depth distance is set at each of the sensors 20A, 20B, 20C.

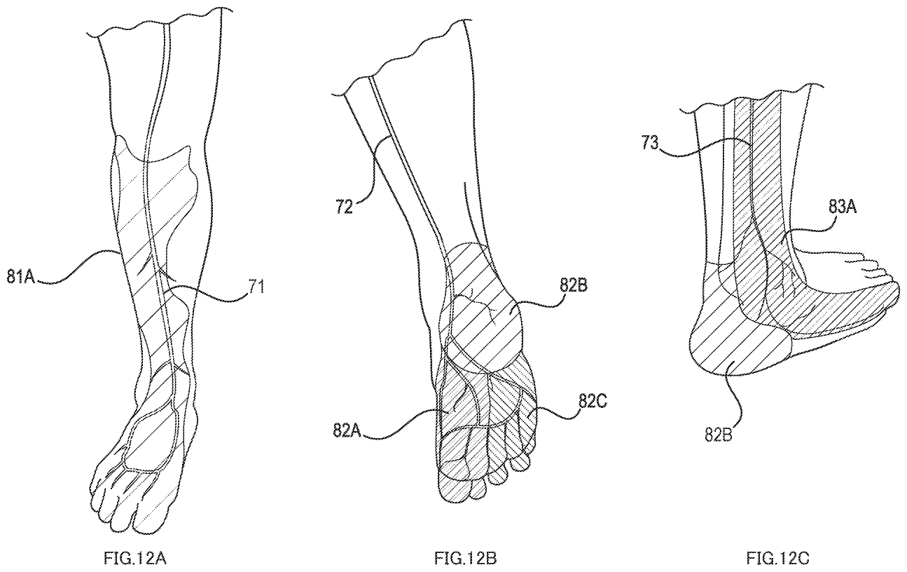

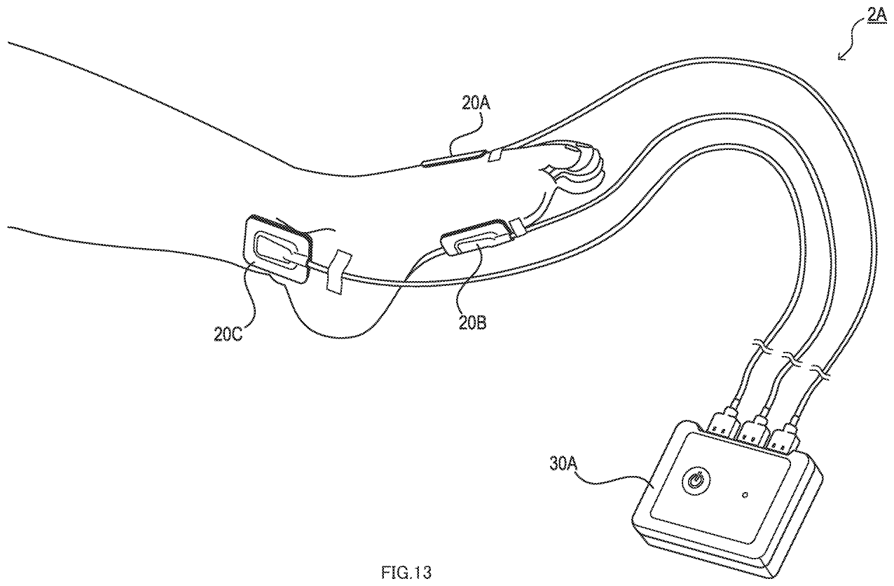

First, the sensors 20A, 20B, 20C are connected to the processing unit 30A. Then, the sensors 20A, 20B, 20C are each arranged and fixed on a skin in a portion of a measurement target region where measurement will be performed. In the present embodiment, as shown in FIG. 13, the sensor 20A is arranged and fixed on a top, specifically a dorsum, of a foot of the patient. The sensor 20B is arranged and fixed on a sole, specifically a bottom, of the same foot of the patient. The sensor 20C is arranged and fixed on a lateral ankle portion, specifically a lateral malleolus, of the same foot of the patient. The lateral malleolus here means a portion, including a lateral ankle of a foot and an area surrounding the ankle, such as an area 83A surrounding the ankle in FIG. 12C. The sensor 20A arranged on the dorsum of the foot is an example of a foot dorsum sensor. The sensor 20B arranged on the bottom of the foot is an example of a foot bottom sensor. The sensor 20C arranged on the lateral malleolus is an example of a lateral malleolus sensor.

Generally, a patient with lower limb arteriosclerosis obliterans has insufficient blood flow in the peripheral part of the lower limb, for example, due to narrowing or occlusion occurred in a blood vessel, and thus the tissue oxygen saturation in the peripheral part of the lower limb is low. On the other hand, when the causative narrowing or occlusion is improved by treatment, the blood flow in the peripheral part is increased, and the increase in blood flow leads to an increased tissue oxygen saturation. That is, by arranging the sensor 20 in the peripheral part of the lower limb, changes in blood flow of the lower limb as a result of treatment can be observed.

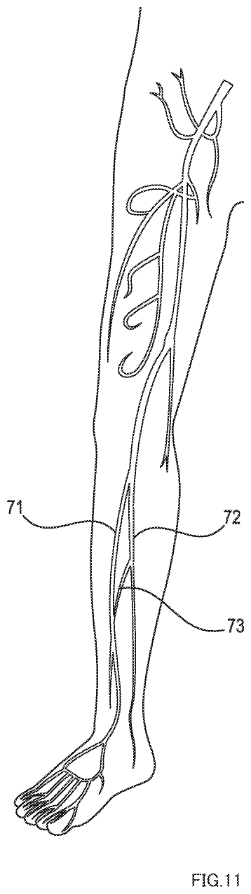

Generally, in the peripheral part of the lower limb, blood is perfused through three different arteries to respective areas, as shown in FIG. 11. More specifically with reference to FIG. 12A to FIG. 12C, an anterior tibial artery 71, a posterior tibial artery 72, and a peroneal artery 73 run in the peripheral part of the lower limb, i.e., in a lower leg. It is known that the anterior tibial artery 71 perfuses blood mainly to an area on a foot top side, specifically an area 81A on a foot dorsum side. It is known that the posterior tibial artery 72 perfuses blood mainly to an area on a sole side, specifically areas 82A, 82B, and 82C on a foot bottom side. It is known that the peroneal artery 73 perfuses blood mainly to an area surrounding the lateral ankle of the foot, specifically an area 83A surrounding the lateral malleolus.

By arranging the sensors 20A, 20B, and 20C as described above and measuring respective tissue oxygen saturation information, it is possible to estimate changes in blood flow in the blood vessels that perfuse blood to the respective areas. More specifically, in a case where there is a change in value of the tissue oxygen saturation information of the channel to which the sensor 20A arranged on the foot dorsum is connected, it can be estimated that mainly the blood flow in the anterior tibial artery 71 has changed. In a case where there is a change in value of the tissue oxygen saturation information of the channel to which the sensor 20B arranged on the foot bottom is connected, it can be estimated that mainly the blood flow in the posterior tibial artery 72 has changed. In a case where there is a change in value of the tissue oxygen saturation information of the channel to which the sensor 20C arranged on the lateral malleolus is connected, it can be estimated that mainly the blood flow in the peroneal artery 73 has changed. That is, it is possible to estimate through which blood vessel the blood flow has changed as a result of treatment by revascularization, based on the changes in the tissue oxygen saturation information of the respective areas.

After the sensors 20 are arranged, the power button 31 is operated to turn on power of the processing unit 30. When power of the processing unit 30 is turned on, power is supplied also to the sensor 20, and collection of optical information is started.

When collection of optical information is started, tissue oxygen saturation information is calculated for the respective channels by the arithmetic processor 53, and calculation results are displayed on the display/operation section 64. Specifically, measured tissue oxygen saturation values and other information are displayed on specified areas of the display/operation section 64 for the respective channels. In the present embodiment, a description will be given of an example case where blood flow in the peripheral part of the lower limb is insufficient and the tissue oxygen saturation value is low before treatment is started. Specifically, a description will be given of an example case where the tissue oxygen saturation values of the respective channels are each less than 50% before treatment is started, while generally a normal value of the tissue oxygen saturation is approximately 55 to 65%.

Subsequently, treatment by a doctor is performed. The doctor performs revascularization by endovascular treatment in which a catheter is moved to a part of a blood vessel with occlusion or narrowing, and a balloon at a leading end of the catheter is expanded or a metal casting called a stent is left in place, to thereby eliminate the occlusion or the narrowing of the blood vessel, or performs revascularization by bypass creation in which blood flow is restored through another direct route circumventing the occlusion or the narrowed part of the blood vessel. Once blood flow is restored by the aforementioned treatment, the blood flow condition in the peripheral part of the lower limb is improved. That is, blood flow in the peripheral part of the lower limb is increased. The increase in blood flow in the peripheral part of the lower limb leads to an increased tissue oxygen saturation value. In a case where the blood flow condition in a distal end of the lower limb is not improved, specifically in a case where blood flow in the peripheral part of the lower limb is not increased, the doctor again performs treatment by moving the catheter to a different part of the blood vessel, which assumes to have occlusion, to thereby eliminate the occlusion in the different part. The doctor repeats the aforementioned operation to perform treatment.

The notifier 55 performs a process of sequentially comparing the tissue oxygen saturation value calculated by the arithmetic processor 53 and a stored value of the threshold. If the number of channels each having a value of the tissue oxygen saturation equal to or greater than the threshold is two or more, the notifier 55 outputs a notification signal notifying that to the display device 6.

In the present embodiment, a description hereinafter will be given of an example case where blood flow in the anterior tibial artery 71 is improved by a first procedure by the doctor, and blood flow in the posterior tibial artery 72 is improved by a subsequent procedure. Places in which blood flow is improved and the order of such improvement are only examples for description, and are not limited to the aforementioned.

Improvement in blood flow in the anterior tibial artery 71 by the first procedure leads to an increased blood volume in the area where blood is perfused mainly by the anterior tibial artery 71. Then, the tissue oxygen saturation value of the channel to which the sensor 20A arranged on the foot dorsum side is connected increases.

When the tissue oxygen saturation value of the channel to which the sensor 20A is connected increases to 50% or greater, the notifier 55 detects that the tissue oxygen saturation value of the channel of the sensor 20A has become equal to or greater than the threshold. In this case, the number of channels each having a value of the tissue oxygen saturation equal to or greater than the threshold is one, and thus the notifier 55 does not output a notification signal.

Subsequently, treatment of another area by the doctor leads to an improved blood flow in the posterior tibial artery 72. Then, the tissue oxygen saturation value of the channel to which the sensor 20B arranged on the foot bottom side is connected increases.

When the tissue oxygen saturation value of the channel to which the sensor 20B is connected increases to 50% or greater, the notifier 55 detects that the tissue oxygen saturation value of the channel to which the sensor 20B is connected has become equal to or greater than the threshold. In this case, the number of channels of the tissue oxygen saturation each having a value equal to or greater than the threshold is two, the notifier 55 outputs a notification signal notifying that. Specifically, the notifier 55 sends a notification signal to the display device 6 through the communicator 51.

Upon receipt of the notification signal, the display device 6 displays a notification that the number of channels each having a value equal to or greater than the threshold has become two or more on a specified area of the display/operation section 64.

When the number of channels of the tissue oxygen saturation each having a value equal to or greater than the threshold is three, the notifier 55 outputs a notification signal notifying that the number of channels of the tissue oxygen saturation each having a value equal to or greater than the threshold is three. Upon receipt of the notification signal from the notifier 55, the display device 6 displays a notification that the number of channels each having a value equal to or greater than the threshold has become three on the display/operation section 64.

When receiving the notification signal, the display device 6 may display, on the display/operation section 64, tissue oxygen saturation values of the channels equal to or greater than the threshold in a different color for emphasis, to thereby notify the user that the number of channels each having a value equal to or greater than the threshold has become two or more. Alternatively, when receiving the notification signal, the display device 6 may display a pop-up window or the like, or output a sound or the like, to thereby notify the user that the number of channels each having a value equal to or greater than the threshold has become two or more. Also alternatively, the display device 6 may notify the user that the number of channels each having a value equal to or greater than the threshold has become two or more by other display methods.

The doctor determines whether the treatment is sufficient based on the tissue oxygen saturation information calculated by the arithmetic processor 53, the notification by the notifier 55, and other biological information of the patient and information regarding findings, and other information, and then finishes the surgery.

3. Results of Clinical Study

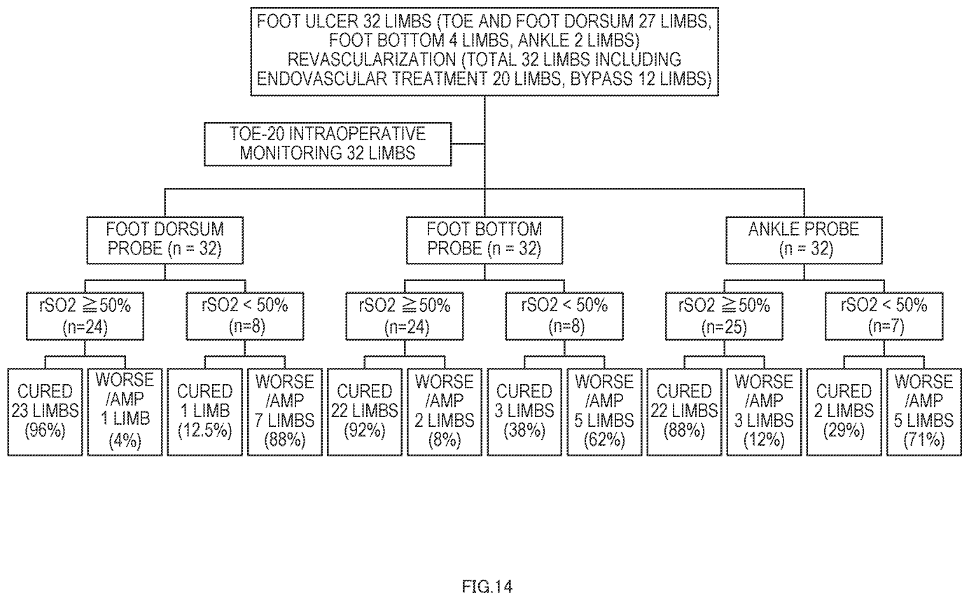

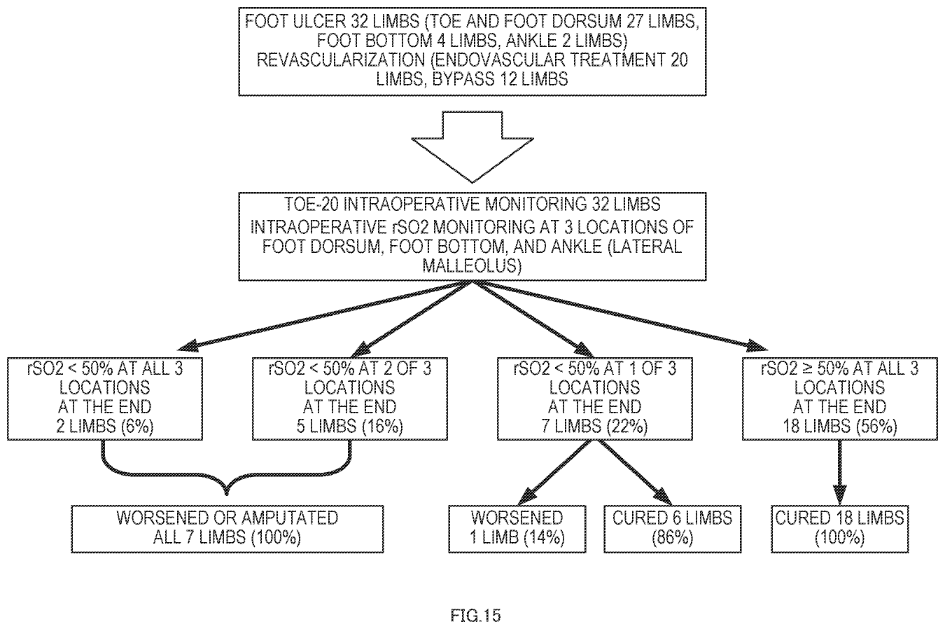

FIG. 14 and FIG. 15 show results of revascularization (32 limbs) performed on severe ischemic limbs with skin ulcers due to arteriosclerosis obliterans of lower limbs. During the treatment, the biological information collection device 1A was used to measure tissue oxygen saturations of specified areas of a treatment target limb of each patient. More specifically, the sensors 20A, 20B, and 20C were arranged on a dorsum of foot, a bottom of foot, and a lateral malleolus of the patient, as shown in FIG. 13, and measurement was performed regarding the tissue oxygen saturation information of the respective areas during the operation.

In this clinical study, 18 limbs, which is 56% of the entire limbs, had a tissue oxygen saturation value of 50% or greater at the end of treatment in the three areas where the sensors 20 were arranged. In this case, each patient had a good prognosis, and all the 18 limbs were cured, resulting in a cure rate of 100%. Also, 7 limbs, which is 22% of the entire limbs, had a tissue oxygen saturation value of 50% or greater in two of the three areas where the sensors 20 were arranged. In this case, 6 limbs were cured, resulting in a cure rate of 86%.

On the other hand, 5 limbs, which is 16% of the entire limbs, had a tissue oxygen saturation value of less than 50% at the end of treatment in two areas, and 2 limbs, which is 6% of the entire limbs, had a tissue oxygen saturation value of less than 50% in all the three areas. In these cases, all the patients had a poor prognosis, resulting in worsened ulcer or limb amputation in all cases.

From the results of the clinical study above, it can be determined that it is possible to estimate the prognosis after treatment by a comprehensive analysis of the tissue oxygen saturations in the dorsum of foot, the bottom of foot, and the lateral malleolus at the end of revascularization that are measured using the biological information collection device 1A. In other words, it can be determined that it is possible to estimate the prognosis after treatment based on the number of areas, among the dorsum of foot, the bottom of foot, and the lateral malleolus, having a tissue oxygen saturation value equal to or greater than the threshold at the end of revascularization.

Specifically, if the tissue oxygen saturations in the specified three areas at the end of revascularization are 50% or greater, it is possible to determine that the blood flow to a treatment target limb is sufficiently restored, and the prognosis will be very good and cure can be expected. That is, it is possible to assume that the blood flows are restored in respective areas to which blood is perfused by the anterior tibial artery, the posterior tibial artery, and the peroneal artery, and to determine that the prognosis of the patient's limb will be very good and cure can be expected.

If the tissue oxygen saturations in any two areas are equal to or greater than the threshold, the blood flow to the treatment target limb is restored to a certain extent, and a further improvement in blood flow condition by, for example, formation of a collateral pathway thereafter can be expected. That is, it is possible to determine that the prognosis of the patient's limb will be good and cure can be expected.

For example, if the tissue oxygen saturation values of the dorsum of foot and the bottom of foot are 50% or greater, it can be assumed that, as a result of the treatment, the blood flows are restored to a certain extent in respective areas to which blood is perfused by the anterior tibial artery and the posterior tibial artery. Also, for example, if the tissue oxygen saturation values of the bottom of foot and the lateral malleolus are 50% or greater, it can be assumed that, as a result of the treatment, the blood flows are restored to a certain extent in respective areas to which blood is perfused by the posterior tibial artery and the peroneal artery. Further, for example, if the tissue oxygen saturation values of the lateral malleolus and the dorsum of foot are 50% or greater, it can be assumed that, as a result of the treatment, the blood flows are restored to a certain extent in respective areas to which blood is perfused by the peroneal artery and the anterior tibial artery. Then, it is possible to determine that restoration of the blood flow in those blood vessels and formation of collateral pathways thereafter will lead to an improved blood flow to the treatment target limb and a cure can be expected.

On the other hand, if the tissue oxygen saturation values of the respective areas at the end of revascularization are less than 50% in all the areas, or less than the threshold in any two areas, it can be estimated that the prognosis will be very poor. Specifically, it can be assumed that the blood flow is not restored in any of the areas to which blood is perfused by the anterior tibial artery, the posterior tibial artery, and the peroneal artery, or that the blood flow is not restored in the areas to which blood is perfused by any two of the anterior tibial artery, the posterior tibial artery, and the peroneal artery. Then, it can be estimated that the prognosis will be very poor since blood flow is not restored and blood flow in the entire foot as the treatment target is not improved.

That is, it is possible to determine that based on the number of areas having tissue oxygen saturations equal to or greater than the threshold, status of restoration of blood flow to the treatment target limb can be assumed, and that the cure rate by revascularization can be estimated. In other words, it is possible to determine that measurement results by the biological information collection device 1A at the end of treatment can be used for estimation of cure by revascularization.

4. Description of Effects

With the biological information collection device 1A and the biological information collection sensor unit 2A configured as described above, respective tissue oxygen saturation information is calculated based on optical information collected by the two or more sensors 20 arranged in respective different areas. Thus, it is possible to simultaneously obtain information regarding status of tissue oxygen saturation and blood flow condition in different areas of a measurement target region. Also, by arranging the sensors 20, for example, considering the state of running of blood vessels that perfuse blood to the measurement target region, it is possible to obtain information regarding tissue oxygen saturation and blood flow condition of the entire measurement target region.

In a case where the number of the sensors 20 that have collected optical information based on which tissue oxygen saturation information equal to or greater than the threshold is calculated satisfies a specified condition, the notifier 55 outputs a notification signal. In other words, when the number of channels of tissue oxygen saturation information equal to or greater than the threshold satisfies the specified condition, the notifier 55 outputs a notification signal. Thus, it is possible to easily recognize that the number of areas, each with a change to a specified extent or more in tissue oxygen saturation information, has reached a specified number. Specifically, it is possible to easily recognize the degree of existence of areas in the measurement target region where there has occurred a certain change in oxygenation state and blood flow condition of the tissue. Then, it is possible to easily recognize overall changes in oxygenation state and blood flow condition of the tissues in the measurement target region.

The biological information collection sensor unit 2A comprises the sensors 20A, 20B, 20C to measure optical information of respective different areas. Thus, the biological information collection sensor unit 2A can simultaneously collect tissue oxygen saturation information of three respective different areas.

In the biological information collection sensor unit 2A, when the number of channels having tissue oxygen saturations equal to or greater than the threshold has become two or more, the notifier 55 outputs a notification signal notifying that. Thus, it is possible to easily recognize that the tissue oxygen saturation information in two or more areas has changed by a specified value or greater.

The biological information collection sensor unit 2A comprises the foot dorsum sensor, the foot bottom sensor, and the lateral malleolus sensor. In other words, measurement can be performed by arranging the sensors 20A, 20B, 20C respectively on the dorsum of foot, the bottom of foot, and the lateral malleolus of the patient's lower limb. Measurement in this manner enables collection of tissue oxygen saturation information in respective areas of the patient's foot. It is generally known that blood is perfused in the dorsum of foot, the bottom of foot, and the lateral malleolus of the lower limb through different blood vessels. Accordingly, measurement by arranging the sensors 20A, 20B, 20C in the aforementioned manner enables obtaining tissue oxygen saturation information in respective areas of the patient's foot in which blood is perfused by different blood vessels, and also enables comprehensive understanding of blood flow conditions and tissue oxygenation of the entire foot. Also, blood flow conditions of the blood vessels that perfuse blood to the respective areas can be assumed.

In general, angiosomes of a lower limb, specifically areas in which blood is perfused by specific blood vessels differ for each patient. Particularly, for example, in case of a patient with arteriosclerosis obliterans, occurrence of narrowing or occlusion in a major blood vessel results in formation of a collateral pathway, and blood flow is performed by the collateral pathway, and thus angiosomes of the patient may be different from angiosomes of an able-bodied person. In light of this, by performing measurement with arrangement of the sensors 20A, 20B, 20C in respective areas where blood is considered to be perfused by the anterior tibial artery, the posterior tibial artery, and the peroneal artery depending on the status of the patient, for whom measurement is performed, it is possible to obtain information regarding blood flow conditions of the respective blood vessels, specifically the anterior tibial artery, the posterior tibial artery, and the peroneal artery. It is also possible to change the arrangement of the sensors depending on the patient or on the site of ulcer. For example, it is possible to perform measurement with the sensors 20A, 20B, 20C arranged on a foot dorsum, a heel, and a medial malleolus. Then, it is possible to obtain tissue oxygen saturation information of areas in which blood is perfused by respective blood vessels.

Use of the biological information collection device 1A during revascularization of a lower limb enables sequential monitoring of changes in tissue oxygen saturation information in different areas of the lower limb of a patient. Then, based on the changing state of tissue oxygen saturation information, changes in blood flow in respective areas of the lower limb of the patient can be recognized. Further, it can be expected to estimate the possibility of cure of the target region after revascularization based on the number of channels each having a tissue oxygen saturation equal to or greater than the threshold, specifically based on the number of areas each having a tissue oxygen saturation equal to or greater than the threshold. That is, it can be expected to use a notification by the notifier 55 as information usable for determining effectiveness of a treatment performed to improve blood flow or prognosis of a patient.

Specifically, use of the biological information collection device 1A during revascularization of a lower limb enables giving an objective indication for revascularization that has been performed conventionally based on experiences of doctors. A doctor can determine whether the performed treatment of revascularization is sufficient based on the notification by the notifier 55 and the tissue oxygen saturation information of respective areas calculated by the biological information collection sensor unit 2A. Specifically, measurement results and notifications by the biological information collection device 1A can be used for determining effectiveness of the treatment by revascularization and the prognosis of a patient. Also, it can be expected, for example, to avoid finishing a surgery with insufficient treatment performed, or to avoid continuing excessive treatment that causes excessive burden to a patient. Further, for a patient who has undergone a surgery that was inevitably finished with insufficient treatment performed, it will be possible to minimize disorder of the patient by taking second-best measures early, including treatment other than revascularization, such as medical therapy and physical therapy.

In the embodiment above, the description has been given of an example in which, when the number of channels each having a value equal to or greater than the threshold is two or more, the notifier 55 outputs a notification signal to notify that. The notifier 55 may output information regarding the prognosis of a patient after revascularization. For example, the notifier 55 may output information regarding treatment effects and prognosis of revascularization. In a specific example case, the notifier 55 may output a notification signal indicating that the prognosis of the patient, for whom revascularization is being performed, is highly likely to be good when the number of channels each having a value equal to or greater than the threshold is two or more.

Alternatively, information may be output regarding estimated cure rate after revascularization of the measurement target region depending on the number of channels each having a value equal to or greater than the threshold. For example, information may be output regarding an estimated cure rate of ischemic ulcer or wound based on results of the aforementioned clinical study.

For example, the notifier 55 may output a signal to cause display indicating an estimated cure rate of 100% when the number of channels each having a value equal to or greater than the threshold is three. The notifier 55 may output a signal to cause display indicating an estimated cure rate of, for example, 86% when the number of channels each having a value equal to or greater than the threshold is two. The display device 6 may display, on the display/operation section 64, information regarding the received estimated cure rate as well as notification of the number of channels each having a value equal to or greater than the threshold. Alternatively, the notifier 55 may output a signal to cause display of information regarding an estimated rate of lower limb amputation, or may output a signal to cause display of possibility of a poor prognosis when the number of channels each having a value equal to or greater than the threshold is a specified number or less. This enables determination as to whether performed treatment of revascularization is sufficient more objectively and easily.

In the description above, the display device 6 may perform a process of selecting an estimated cure rate based on the number of channels that is notified and to cause the display/operation section 64 to display the estimated cure rate. For example, it may be configured such that the display device 6 stores, in its storage, a table in which the numbers of channels each having a value equal to or greater than the threshold and corresponding estimated cure rates linked to the respective numbers. Then, when a notification signal is received from the notifier 55, the display device 6 may perform a process of selecting an estimated cure rate by referring to the table based on the number of channels that is notified, and to cause the display/operation section 64 to display the estimated cure rate.

It may be configured such that the value of the estimated cure rate to be displayed based on the number of channels each having a value equal to or greater than the threshold is appropriately updated to the latest value based on actual treatment result data that is stored. For example, the value of the estimated cure rate to be displayed may be updated by a user's operation of the display device 6. Alternatively, the value may be updated automatically through periodical access to an external server or the like, in which actual treatment result data is stored, by the biological information collection sensor unit 2A or the display device 6.

As the estimated cure rate to be displayed, values may be employed that are calculated by an AI, other computer systems, and the like based on results of actually performed treatment and information of multivariate analysis with other factors, including presence/absence of complications, such as diabetes or high-blood pressure, age, and lifestyle information, such as history of smoking.

The notifier 55 may output information regarding finish of revascularization depending on the number of channels each having a value equal to or greater than the threshold. Specifically, the notifier 55 may output a signal notifying the user that necessary and sufficient treatment effects have been obtained and the treatment can be finished when the number of tissue oxygen saturations equal to or greater than the threshold becomes two or more.

In the embodiment above, the description has been given of a configuration in which the notifier 55 outputs a notification signal when the number of channels each having a value equal to or greater than the threshold is two or more. Alternatively, for example, the notifier 55 may be configured to output a notification signal when the number of channels each having a value equal to or greater than the threshold is two or less. With such notification, it is possible to easily recognize that the tissue oxygen saturation values in two or more areas of the measurement target region are low. For example, it is possible to easily recognize that the entire blood flow conditions in the measurement target region are poor.

In the embodiment above, the description has been given of a case of using the biological information collection device 1A during revascularization of a lower limb. The biological information collection device 1A may be used during, for example, revascularization of an upper limb. The biological information collection device 1A may also be used, for example, for monitoring of a patient during treatment of other regions. Further, information that is output by the biological information collection device 1A may be used for determining effectiveness of other treatments.

In the embodiment above, the description has been given of an example where the notifier 55 sequentially compares the threshold and the tissue oxygen saturation information calculated by the arithmetic processor 53. Alternatively, it may be configured such that, for example, at a time when the user provides instructions, the notifier 55 compares the threshold and tissue oxygen saturation information calculated by the arithmetic processor 53, and outputs a notification signal when specified conditions are satisfied. With this configuration, a process by the notifier 55 can be performed when needed by the user.

In the embodiment above, the description has been given of an example where the same threshold is set for each of the channels. Alternatively, it may be configured such that respective different thresholds are set for the sensors 20. For example, respective different thresholds may be set for the area where blood is perfused mainly by the anterior tibial artery, the area where blood is perfused mainly by the posterior tibial artery, and the area where blood is perfused mainly by the peroneal artery. Also, it may be configured such that the notifier 55 outputs a notification signal when the tissue oxygen saturations of any two areas become equal to or greater than the respective thresholds. With this configuration, it is possible to provide a notification considering, for example, differences in influence due to the blood vessels that perfuse blood to the measurement target region.

Alternatively, it may be configured such that respective thresholds are automatically set by the arithmetic processor 53 or the notifier 55 according to respective identification signals of the sensors 20. For example, it is previously specified that the sensor 20A is a sensor for a foot dorsum area, the sensor 20B is a sensor for a foot bottom area, and the sensor 20C is a sensor for a lateral malleolus area. It may be configured such that when the sensors 20 are connected, the arithmetic processor 53 sets respective thresholds according to the identification information of the sensors 20. With this configuration, it is possible to set respective thresholds for the sensors 20 by a simple method.

It may be configured such that the sensors 20 are set to respective depth distances corresponding to areas in which measurement is performed. For example, it may be configured such that the depth distance of the sensor 20A for measuring the foot dorsum area is set to a short distance, and the depth distance of the sensor 20B for measuring the foot bottom area is set to a long distance. Also, it may be configured such that a measurement depth distance of the sensor 20C for measuring the lateral malleolus area is set to, for example, a distance between respective measurement depth distances of the sensors 20A and 20B. It may also be configured such that the arithmetic processor 53 calculates tissue oxygen saturation information according to the respective identification signals of the sensors 20. With this configuration, measurement according to the depth of the measurement target tissue is performed, and it is possible to obtain more accurate tissue oxygen saturation information of a target region. It is thus possible to measure, for example, changes in blood flow in each measurement target region more accurately.

The arithmetic processor 53 or the notifier 55 may be configured to automatically set a threshold according to the tissue oxygen saturation information at the time of starting measurement. For example, the arithmetic processor 53 or the notifier 55 may be configured to set, as a threshold, a value obtained by adding a specified value to the tissue oxygen saturation value at the time of starting measurement. Alternatively, the arithmetic processor 53 or the notifier 55 may be configured to set a threshold referring to, for example, a table or the like in which tissue oxygen saturation values at the time of starting measurement and thresholds corresponding to the respective values are linked and recorded. The storage 54 may store the table. With such configuration, it is possible to easily set an appropriate threshold according to the tissue oxygen saturation information at the time of starting measurement.

In the embodiment above, the description has been given of an example where the biological information collection sensor unit 2A comprises the three sensors 20. The biological information collection sensor unit 2A may comprise, for example, four or more sensors 20, or the biological information collection sensor unit 2A may comprise two sensors 20. In the embodiment above, the description has been given of an example where the notifier 55 outputs a notification signal when the number of channels each having a tissue oxygen saturation equal to or greater than the threshold is two or more. Alternatively, it may be configured to output a notification signal when the number of channels each having a tissue oxygen saturation equal to or greater than the threshold is three or more. Alternatively, it may be configured to output a notification signal when the number of channels each having a tissue oxygen saturation equal to or greater than the threshold becomes a preset number depending on the number of the sensors 20 that are connected. With such configuration, the biological information collection sensor unit 2A can collect tissue oxygen saturation information of various measurement target regions, and also output a notification signal depending on the number of the sensors 20 that are connected.

When outputting a notification signal, the notifier 55 may also output a signal indicating in which area the sensor 20 of the channel having a value equal to or greater than the threshold is arranged. For example, assume that the sensor 20A arranged on the foot dorsum and the sensor 20B arranged on the foot bottom have collected optical information of tissue oxygen saturation information equal to or greater than the threshold. In this case, the notifier 55 may output, along with a notification signal, a signal indicating that the sensors 20 related to the notification are the sensors 20 arranged on the foot dorsum and the foot bottom. Such configuration facilitates determination considering positions of the sensors 20.

The notifier 55 may be configured to provide a notification signal also considering information regarding arrangement positions of the sensors 20 of the channels each having a value equal to or greater than the threshold. For example, it may be configured such that a notification signal is not output even if the number of channels each having a tissue oxygen saturation equal to or greater than the threshold is a specified number or more, unless the tissue oxygen saturation value of the channel connected with the sensor 20 arranged in a specific area is equal to or greater than the threshold. More specifically, for example, it may be configured not to output a notification signal even if the number of channels each having a tissue oxygen saturation equal to or greater than the threshold is two, unless the channel connected with the sensor 20 arranged on the foot dorsum is included. Alternatively, it may be configured to output a notification signal when the tissue oxygen saturation value of the channel connected with the sensor 20 arranged in a specific area is equal to or greater than the threshold, even if the number of channels each having a tissue oxygen saturation equal to or greater than the threshold is less than a specified number. For example, it may be configured to output a notification signal when the tissue oxygen saturation value of the channel connected with the sensor 20 arranged on the foot dorsum is equal to or greater than the threshold, even if the number of channels each having a tissue oxygen saturation equal to or greater than the threshold is one. With such configuration, a notification signal is output considering the area where the sensor 20 is arranged. Thus, it can be expected to provide more accurate information responding to the condition of the measurement target region.

It may be configured such that weighting is performed for the respective sensors 20 to calculate scores for notification, and the notifier 55 performs notification based on the result of calculated scores. For example, a score “1.5” is given when the tissue oxygen saturation of the channel of the sensor 20 arranged on the foot bottom is equal to or greater than the threshold, and a score “0.8” is given when the tissue oxygen saturation of the channel of any other sensor 20 is equal to or greater than the threshold. The notifier 55 may be configured to output a notification signal when the total of the scores reaches 1.6 or more. The aforementioned score values are exemplary, and these values are not limitative. With such configuration, it is possible to perform notification, for example, based on a clinical condition of an area to be measured in the measurement target region.

The present disclosure is applicable not only to the aforementioned embodiments, but also to any embodiment obtained by suitably combining the aforementioned embodiments, and thus is not limitative.

What is claimed is:

1. A biological information collection sensor unit comprising:
two or more sensors,
the sensors each comprising:
a light emitter to emit near-infrared light or infrared light; and
a light receiver to collect transmitted light including at least light that has transmitted through a measurement target tissue, and
the sensors being arranged on surfaces of a measurement target region and configured to collect optical information regarding tissues of the measurement target region; and
a processor configured to calculate tissue oxygen saturation information regarding oxygen saturation of the tissues based on the optical information,
wherein the two or more sensors are sensors to be arranged in respective different areas of the measurement target region, and
wherein the processor comprises:
an arithmetic processor configured to calculate the tissue oxygen saturation information for the respective sensors; and
a notifier configured to output a notification signal when the number of the sensors that have collected the optical information used to calculate the tissue oxygen saturation information equal to or greater than a preset threshold satisfies a specified condition.

2. The biological information collection sensor unit according to claim 1,
wherein the sensors comprise:
a first sensor configured to collect the optical information of a first area of the measurement target region;
a second sensor configured to collect the optical information of a second area, which is different from the first area, of the measurement target region; and
a third sensor configured to collect the optical information of a third area, which is different from the first area and the second area, of the measurement target region.

3. The biological information collection sensor unit according to claim 2,
wherein the notifier outputs the notification signal when the number of the sensors that have collected the optical information used to calculate the tissue oxygen saturation information equal to or greater than the threshold is two or more.

4. The biological information collection sensor unit according to claim 2,
wherein the measurement target region is a lower limb of a patient,
wherein the first sensor is a foot dorsum sensor to be arranged on a foot dorsum area,
wherein the second sensor is a foot bottom sensor to be arranged on a foot bottom area, and
wherein the third sensor is a lateral malleolus sensor to be arranged on a lateral malleolus area.

5. The biological information collection sensor unit according to claim 2,
wherein the measurement target region is a lower limb of a patient,
wherein the first area is a portion in which blood is perfused by an anterior tibial artery,
wherein the second area is a portion in which blood is perfused by a posterior tibial artery, and
wherein the third area is a portion in which blood is perfused by a peroneal artery.

6. The biological information collection sensor unit according to claim 4,
wherein the biological information collection sensor unit is a sensor unit to be used to collect the tissue oxygen saturation information of the lower limb during revascularization of the lower limb, and
wherein the notifier is configured to output information regarding prognosis of the patient after the revascularization based on the number of the sensors that have collected the optical information used to calculate the tissue oxygen saturation information equal to or greater than the threshold.

7. A biological information collection device comprising:
the biological information collection sensor unit according to claim 1; and
a display configured to display the tissue oxygen saturation information.

8. A biological information processing unit for use in a biological information collection sensor unit to obtain tissue oxygen saturation information regarding oxygen saturation of a measurement target tissue,
the biological information processing unit comprising:
an arithmetic processor configured to calculate the tissue oxygen saturation information for respective two or more sensors based on respective optical information output by the sensors, the sensors each comprising: a light emitter to emit near-infrared light or infrared light; and a light receiver to collect transmitted light including at least light that has transmitted through the measurement target tissue, and the sensors being arranged on surfaces of respective different areas of a measurement target region and configured to collect information regarding tissues of the measurement target region; and
a notifier configured to compare a preset threshold and the respective calculated tissue oxygen saturation information and to output a notification signal when the number of the sensors that have collected the optical information used to calculate the tissue oxygen saturation information equal to or greater than the threshold satisfies a specified condition.
